(19) United States
(12) Patent Application Publication
(10) Pub. No.: US 2024/0047041 A1
(43) Pub. Date: Feb. 8, 2024

(54) SYSTEMS AND METHODS FOR TREATING DEPRESSION USING A DIGITAL THERAPEUTIC

(71) Applicants: OTSUKA AMERICA PHARMACEUTICAL, INC., Rockville, MD (US); Click Therapeutics, Inc., New York, NY (US)

(72) Inventors: Ainslie FORBES, Princeton, NJ (US); John DOCHERTY, Princeton, NJ (US); Brian IACOVIELLO, New York, NY (US); Daniel Clifton CARPENTER, Monroe, CT (US); Monica Jane WARD, Toronto (CA)

(73) Assignees: Otsuka America, Inc., San Francisco, CA (US); Click Therapeutics, Inc., New York, NY (US)

(21) Appl. No.: 18/269,271

(22) PCT Filed: Jan. 5, 2022
(86) PCT No.: PCT/US2022/011328

§ 371 Date: Jun. 22, 2023

Related U.S. Application Data
(60) Provisional application No. 63/176,697, filed on Apr. 19, 2021.
(60) Provisional application No. 63/134,099, filed on Jan. 5, 2021.

(51) Int. Cl. G16H 20/70; G16H 10/20; G16H 40/67; G16H 50/30

(52) U.S. Cl. CPC G16H 20/70; G16H 10/20; G16H 40/67; G16H 50/30

(57) ABSTRACT

Systems and methods for treating depression via a digital therapeutic. The digital therapeutic may be configured to provide memory task exercises according to a schedule and provide psychotherapy lessons according to another schedule. The psychotherapy lessons may include animated videos configured to provide therapeutic intervention through at least one of emotion regulation, behavioral activation and cognitive restructuring. The two schedules may define a six-week treatment period. The memory task exercises may include emotional face memory task (EFMT) exercises and the psychotherapy lessons may include cognitive behavioral therapy (CBT) lessons.

### CROSS-REFERENCE TO RELATED APPLICATIONS

This application claims priority under 35 U.S.C. § 119(e) to U.S. Patent Application Ser. No. 63/176,697, filed on Apr. 19, 2021, and U.S. Patent Application Ser. No. 63/134,099, filed on Jan. 5, 2021, the entire contents of which are hereby incorporated by reference.

### BACKGROUND

Mood disorders may be characterized as a general mental or emotional state that is distorted or inconsistent with an individual's actual circumstances. Depending on the severity, mood disorders may interfere with the individual's ability to function, both professionally and socially. Examples of mood disorders include major depressive disorder (MDD), bipolar disorder, seasonal affective disorder, cyclothymic disorder, persistent depressive disorder (dysthymia), disruptive mood dysregulation disorder, depression related to medical illness, and depression induced by substance use or medication.

An individual diagnosed with a mood disorder involving MDD, for example, may have a persistently low or depressed mood, a decreased interest in pleasurable activities, feelings of guilt or worthlessness, lack of energy, poor concentration, appetite changes, psychomotor agitation, sleep disturbances, or suicidal thoughts. These symptoms can have a severe impact on the individual's overall wellness. Indeed, MDD is considered a leading cause of disability worldwide in terms of total years lost due to disability. Some studies suggest that depression related expenditures in the US may total hundreds of billions of dollars, with employers incurring substantial direct medical costs as well as substantial losses due to absenteeism, presenteeism, and disability. While treatment options for depression exist, there is a continued need to improve treatment outcomes. Additionally, many patients diagnosed with mood disorders, such as MDD, do not receive adequate treatment due to various barriers to accessing standard of care.

### SUMMARY

The disclosed subject matter includes systems and methods for providing therapeutic content via a digital therapeutic (“DTx”) for the treatment of mood disorders, such as depression and, in particular, major depressive disorder (MDD). The therapeutic content may include memory task exercises, psychotherapy lessons, and/or other content. The memory task exercises may include sequentially displaying two or more expression images to a patient receiving treatment for depression. The expression images may be configured to depict certain emotions. The patient may be prompted to determine whether the respective emotions depicted by the displayed expression images match one another. A patient response may be received indicating whether the respective emotions match.

The psychotherapy lessons may be encoded as content, such as videos (in particular, animated videos), audio (such as song, narratives, etc.), haptic content, or other forms of content. The content of the psychotherapy lessons may be configured to provide therapeutic intervention through at least one of emotion regulation (ER), behavioral activation (BA), or cognitive restructuring (CR).

According to an embodiment, the therapeutic content may be provided according to a treatment schedule. The treatment schedule may define a treatment period (duration of treatment) and timing (when to provide) the digital therapeutic. For example, the memory task exercises may be provided according to a treatment schedule. Likewise, the psychotherapy lessons may be provided according to a treatment schedule, which may be the same as or separate from the treatment schedule for the memory task exercises. In other words, a single treatment schedule may define the duration and/or timing for both the memory task exercises and the psychotherapy lessons or a first treatment schedule may define a first duration and timing for the memory task exercises and another, second, treatment schedule may define a second duration and timing for the psychotherapy lessons.

The treatment schedule of memory task exercises and psychotherapy lessons (whether a single schedule or separate schedules) may define a particular treatment period, such as, for example, a six-week treatment period.

In one or more embodiments, the memory task exercises may include emotional face memory task (EFMT) exercises and the psychotherapy lessons may include cognitive behavioral therapy (CBT) lessons. The EFMT exercises and the CBT lessons may each be provided at various frequencies (such as 3 days per week, on alternating days, and the like) over the course of the treatment period.

### BRIEF DESCRIPTION OF THE DRAWINGS

FIG. 1 is a system diagram of an example of a digital therapeutic for treating depression, in accordance with various embodiments.

FIG. 2 is an example of a process for delivering therapeutic content via the digital therapeutic, in accordance with various embodiments.

FIGS. 3A-3C, 4A, 4, 5A, and 5B are examples of images of psychotherapy lessons rendered by the digital therapeutic, in accordance with various embodiments.

FIG. 6 illustrates an example of a therapy schedule for delivering therapeutic content via the digital therapeutic, in accordance with various embodiments.

FIG. 7 is a diagram of an example of a system for delivering therapeutic content via the digital therapeutic, in accordance with various embodiments.

FIG. 8 is a diagram of examples of system components of one or more devices for delivering therapeutic content via the digital therapeutic, in accordance with various embodiments.

### DETAILED DESCRIPTION

The disclosed subject matter is generally directed to a digital therapeutic (DTx). A DTx may refer to the treatment of disorders such as mood disorders through the use of therapeutic content that may be encoded into computer-readable form.

An individual (also referred to herein as a “patient”) that has been diagnosed with a mood disorder, such as MDD, may have persistent negative feelings and emotions. The condition may affect how the individual feels, thinks, and behaves, which may lead to a variety of emotional and physical problems. The individual may have trouble with normal day-to-day activities, such as work, school, social activities, and/or relationships with others. Symptoms of depression may manifest during periodic episodes, which may occur daily, weekly, monthly, or at other intervals. Symptoms may include, but are not limited to (which is not to imply that other listings are limited), feelings of sadness or hopelessness; angry outbursts, irritability, or frustration; loss of interest or pleasure in recreational activities or hobbies; sleep disturbances, including insomnia or excessive sleep; tiredness and lack of energy; reduced appetite and/or weight loss; increased cravings for food and/or weight gain; anxiety, agitation, or restlessness; slowed thinking, speaking, or body movements; feelings of worthlessness or guilt, fixating on past failures, or self-blame; trouble thinking, concentrating, making decisions, and/or remembering things; frequent or recurrent thoughts of death or suicide; and/or unexplained physical problems.

In accordance with the embodiments disclosed herein, the DTx may include electronic content and/or instructions that program one or more computer devices to administer the electronic content, receive interactions by the patient with the electronic content, and/or perform other operations to treat a mood disorder of the patient. As such, the DTx may be administered to a patient in various ways such as via the one or more computer devices. The one or more computer devices may include an application server, a user device, and/or other devices that are programmed with the DTx or portions thereof.

In particular, the functionality of the DTx described herein may operate at the user device, the application server, or both the treatment device and the application server. The user device may generally be operated by the patient and/or user such as a clinician. In some embodiments, the user device may include a patient's mobile device programmed with the computer program instructions and/or other device. In some embodiments, the user device may access some or all functionality of the DTx via an application programming interface (API) exposed by the application server.

In some embodiments, the DTx is configured to render and process therapeutic content that is specifically tailored to treat mood disorder. The therapeutic content may be tailored individually on a patient-by-patient basis. In some embodiments, the particular therapeutic content to be provided to a patient is obtained from a database of therapeutic content. Depending on the patient's progress, different therapeutic content may be selected and provided to the patient. In some embodiments, the therapeutic content, as well as the feedback provided by the patient to the therapeutic content, may be encrypted to prevent unauthorized access to the patient's confidential health data. Furthermore, certain therapeutic content may be inaccessible to patients at different times. This can ensure that targeted therapeutic content is provided to the patient at appropriate times.

The therapeutic content may include video, audio, haptic, or olfactory elements, either individually or in combination. In addition, the therapeutic content may be interactive, prompting input or responses from the patient. The interactive therapeutic content may further obtain feedback passively, such as by capturing biometric information (such as pulse, blood pressure, pulse-ox, breath rate, etc.) from biometric sensors worn by the patient, monitoring facial expressions (such as using computer vision processing) to perform emotion recognition, performing eye-gaze tracking (such as to determine whether the patient is consuming the therapeutic content), and the like. The therapeutic content may be configured to be delivered to (or consumed by) the patient according to a predefined treatment schedule, which may also be referred to as a “dosing regimen.” The DTx may be prescribed by the patient's health care provider (HCP) and/or be made “over the counter.” The DTx may be implemented in the form of a software app or other type of software module, which may be executed on one or more computing devices, such as a network server, desktop computer, laptop, tablet, smartphone, or other computing device. The therapeutic content may be rendered using output components of the computing devices, such as monitors, touch screens, speakers, and the like.

The therapeutic content of the DTx may be provided as a standalone treatment for a mood disorder, such as MDD, or as an adjunct to other types of treatments, such as antidepressant therapies (ADTs). Exemplary ADTs may include selective serotonin reuptake inhibitors (SSRIs), serotonin and norepinephrine reuptake inhibitors (SNRIs), and norepinephrine and dopamine reuptake inhibitors (NDRIs). SSRIs may include escitalopram, citalopram, fluoxetine, paroxetine, and sertraline. SNRIs may include duloxetine, venlafaxine ER/XR, and desvenlafaxine. NDRIs may include bupropion XL/SR.

Depression is a disorder of altered neural connectivity in the patient. The combination of the memory task exercises and the psychotherapy lessons administered to the patient is designed to target cognitive control networks and emotional processing networks to help restore appropriate neural connectivity. The techniques described herein provide an improvement over existing mood disorder treatments in that the combination treatment targets both improved neural function through memory task exercises and strengthening such improvements through specifically designed psychotherapy lessons that condition the patient to utilize the improved neural function. The combination therapy therefore provides a greater treatment effect than memory task exercises alone. Furthermore, for embodiments in which the DTx is implemented on a patient's device, the DTx may facilitate therapeutic intervention, whether through remote network connectivity between the treatment device and backend application servers or in an offline mode without such remote network connectivity. The DTx may further be provided to patients from all types of socio-economic backgrounds and even to patients who are unable to receive traditional mood disorder treatments. Therefore, individuals from all walks of life may be able to overcome the daily challenges caused by mood disorders.

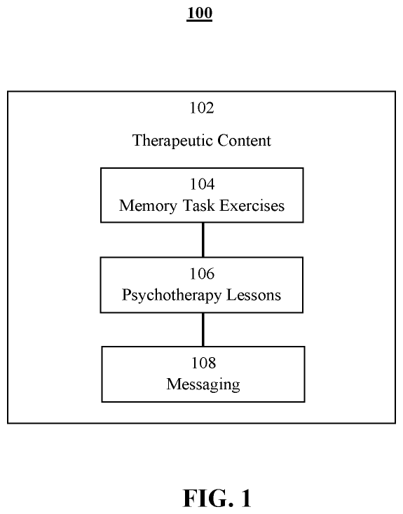

FIG. 1 is a system diagram of an example of a DTx 100 that may include therapeutic content 102 for the treatment of a mood disorder, such as MDD. Therapeutic content 102 may be provided to a patient via a mobile application, API, or other interface. For example, the patient may access a mobile application stored in memory on the patient's user device to administer therapeutic content 102 to the patient. The mobile application may be a native application, an application executed through a web browser, or a web application. In some embodiments, the mobile application may connect the patient's mobile device to a backend server storing data including therapeutic content 102. Various components of therapeutic content 102 may be accessed by the mobile device from the backend server across one or more communications networks (such as the Internet, an Intranet, etc.).

Therapeutic content 102 of DTx 100 may include one or more components, such as memory task exercises 104, psychotherapy lessons 106, and messaging 108. Memory task exercises 104, psychotherapy lessons 106, and/or messaging 108 may be rendered and processed individually or in combination in accordance with a treatment schedule. When used in combination, memory task exercises 104, psychotherapy lessons 106, and/or messaging 108 may function as a synergistic combination therapy for the treatment of depression that provides improvements over traditional mood disorder treatments (such as accessible by a larger subset of the population, less expensive than medication).

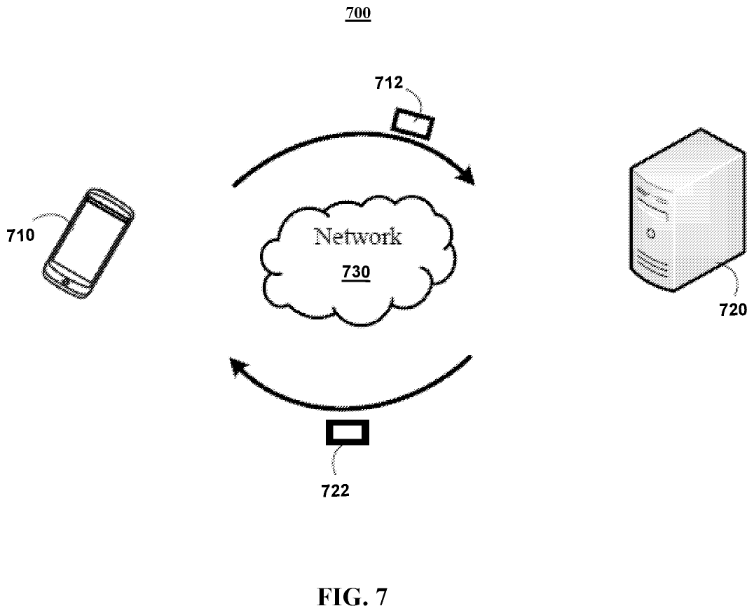

Memory task exercises 104 may target imbalances between the patient's hyperactive emotion-processing regions and hypoactive prefrontal regions, which may underly the patient's cognitive control impairments. Memory task exercises 104 may be encoded with interactive digital content and tasks to be performed by the patient in relation to the DTx 100. The memory task exercises 104 may therefore refer to interactive digital content and operations to prompt the user to perform the tasks and receive inputs from the user relating to the tasks. To illustrate, the memory task exercises 104 will be described in the context of a user device (such as user device 710 illustrated in FIG. 7) that provides the memory task exercises 104. The digital interactive content may include a series of images with respective visual elements. The memory task exercises 104 may further include instructions that prompt the patient to recall whether the visual elements in a presently displayed image matches the visual elements in a previously viewed image. As an example, a first image depicting a first emotion may be displayed, via a user interface rendered on the patient's mobile device, followed by a second image depicting a second emotion (which may be the same or different than the first emotion), followed by a third image depicting a third emotion (which may be the same or different than the first and/or second emotions), and so on. The digital media may then request that, subsequent to the third image being displayed, that the patient identify whether the third image depicts a same (or similar) emotion as that of the first or second image. In other words, the digital media may request that the patient determine whether the N-th image depicts a same or similar emotion as that of the (N-M) image. This form of memory task exercises 104 is referred to as an N-back memory task.

The process of identifying the visual elements in a respective image may trigger the patient's amygdala. The process of recalling whether currently viewed visual elements are the same or different from what was previously viewed may engage the patient's dorsolateral prefrontal cortex by exercising cognitive control abilities. Accordingly, memory task exercises 104 may act to repair the dysfunctional brain circuitry and/or restore neural connectivity by strengthening certain portions of the patient's neural functionality. Memory task exercises 104 may serve as an intervention step in the treatment process, functioning to perform cognitive-emotional training to repair non-functioning or mis-functioning brain circuitry.

In some embodiments, memory task exercises 104 may be encoded as digital media to be administered to and interacted by a patient (such as using the patient's mobile device). For instance, computer program instructions may encode memory task exercises 104, and the computer program instructions may be executed by a computing device. Different memory task exercises may be provided to the patient by accessing different data. For example, data representing first computer program instructions effectuating a first memory task exercise may be accessed via a computing device of a first patient, while data representing second computer program instructions effectuating a second memory task exercise may be accessed via a computing device of a second patient. In some cases, the data can be accessed based on an identifier unique to the patient, such as an IP address, MAC address, and/or serial number associated with the patient's computing device. In some embodiments, the data can be accessed based on log-in credentials provided by the patient (such as username/password, facial recognition authentication, retinal scans, etc.). It should be understood that, as described herein, “accessing,” “retrieving,” and/or “providing” memory task exercises 104 may include accessing, retrieving, and/or providing data including computer program instructions effectuating a given one or more of memory task exercises 104.

Psychotherapy lessons 106 may target symptoms of depression such as all-or-nothing thinking, personalizing, catastrophizing, inactivity, and social isolation. Psychotherapy lessons 106 may address these maladaptive thinking patterns and behaviors by provoking conscious reflection on thought and behavior patterns and the development of alternate behaviors and interpretations of experience. Psychotherapy lessons 106 may also prompt the patient to complete an activity or task. Thus, psychotherapy lessons 106 may be configured to assist the patient to properly utilize or acquire new skills made possible by the repair of dysfunctional circuits and/or the restoration of neural connectivity via the memory task exercises 104. The psychotherapy lessons 106 may further help the patient progressively achieve greater executive control over depressive emotions as they experience the psychotherapy lessons 106 and complete the memory task exercises 104.

Psychotherapy lessons 106 may be provided to patients as digital media, such as, for example, videos (such as animated videos), audio, haptic information, textual information, or other forms of media, or combinations thereof. For example, video data representing a video created to convey a particular psychotherapy lesson to a patient may be accessed by the patient using the patient's computing system. In some cases, the video data may be selected from a database of data representing psychotherapy lessons 106. The video data representing the various psychotherapy lessons may include metadata indicating particular parameters associated with the given psychotherapy lesson. For example, a given video depicting a particular psychotherapy lesson may be tagged with one or more labels indicating what the video includes, a target recipient of the video, a duration of the video, an emotion or set of emotions conveyed by the video, a skill or skills introduced by the video, or other labels, or combinations thereof. In some embodiments, DTx 100 may monitor a progression of a patient through the therapeutic process to determine which psychotherapy lessons 106 have been consumed by the patient to determine which additional psychotherapy lessons 106 are to be provided next to the patient. As described herein, “accessing,” “retrieving,” and/or “providing” psychotherapy lessons 106 may include providing data including computer program instructions to be executed by the patient's computing device to effectuate the particular psychotherapy lesson.

Messaging 108 may be implemented via short message service (SMS), multimedia message service (MMS), push notifications, and the like. Messaging 108 include providing messages to patients, where the messages may be delivered periodically, such as daily, weekly, monthly, etc. The messages provided via messaging 108 may include text, animation, pictures, audio, haptic responses, or other digital media, or combinations thereof. Messaging 108 may derive messages from a library of pre-generated psychotherapy messages and/or a library of pre-generated engagement (reminder) messages. In some embodiments, messaging 108 may include dynamically generating one or more messages using artificial intelligence and/or machine learning techniques, such as via a chatbot. In some embodiments, messaging 108 may implement natural language processing techniques to determine an appropriate message intent, and may select and/or generate a message based on the determined intent.

Messaging 108 may select particular messages to reinforce psychotherapy lessons 106 and may deliver the messages so as to synchronize with the patient's progress through psychotherapy lessons 106. DTx 100 may include logic for selecting and sending messages in accordance with any desired interval (such as 0-4 messages per day). The logic may indicate that a particular message or messages are to be provided to a patient based on a most recently completed memory task exercise and/or psychotherapy lesson, a memory task exercise and/or psychotherapy lesson to be consumed by the patient, input provided by the patient in response to a recent memory task exercise and/or psychotherapy lesson, a request from a clinician or other mental health provider, or based on other criteria. Messaging 108 may include generating and providing a link for the patient to visit one or more psychotherapy lessons 106, which may be previously viewed psychotherapy lessons or new psychotherapy lessons, where skills or strategies for improving a patient's ability to handle difficult moments were, or are to be, conveyed.

Messaging 108 may include providing reminders for the patient to complete memory task exercises 104 and psychotherapy lessons 106 over the course of the treatment schedule. This may include providing a notification to the patient's mobile device to indicate the particular memory task exercise and/or psychotherapy lesson to be completed by the user. The notification may be a displayed prompt (such as a notification message displayed on a display screen of a patient's mobile device), a haptic prompt (such as causing a mobile device of the patient to vibrate to indicate an action to be performed), or may be other notification types. Messaging 108 may also be configured to provide indication of a difficulty, or utility, of completing memory task exercises 104 and/or psychotherapy lessons 106 to further enhance patient engagement and motivation. Messaging 108 may provide messages that are personalized based on the patient's activity, adherence, and/or performance in relation to DTx 100, past messages provided to the patient (or patients with similar attributes (such as age, identified gender, education level)), and the like.

In some embodiments, a particular message to be provided to a patient may be selected using a scoring function configured to score messages (or message components used to generate a message) and rank the messages based on the score. For example, each message may include labels indicating an emotion to be expressed to/by the patient. For instance, there may be n message states, each referring to a particular emotion, thought, condition, etc. of a patient. Based on the memory task exercises and/or psychotherapy lessons (to be) completed by the patient, the scoring function may compute a score representing an optimal message to be provided to the patient based on a patient profile. The patient profile may include a patient profile vector (such as an n-dimensional vector), where each dimension refers to one of the n message states, and having a value representing how appropriate that message state is for the patient. Based on the patient profile vector and vectors representing the messages, the scoring function may determine which message to select (such as a message having highest score). In some embodiments, the values of each attribute of the patient profile vector may be updated based on the memory task exercises and/or psychotherapy lessons completed by the patient, patient feedback provided during the treatment schedule, or other factors.

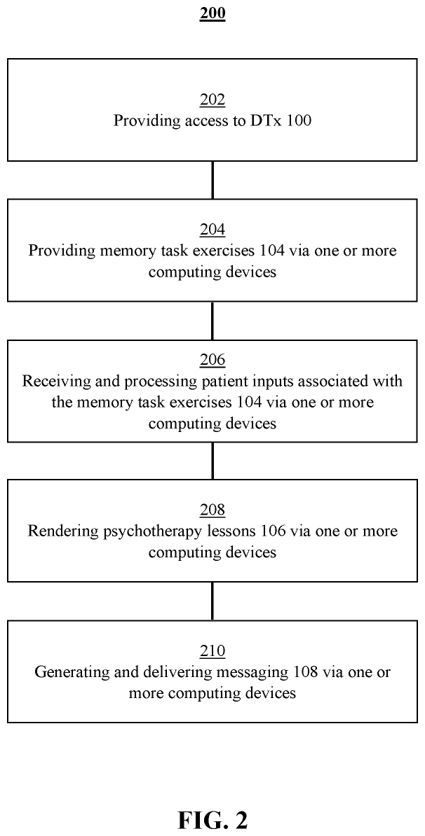

FIG. 2 illustrates an exemplary process 200 for delivering therapeutic content 102 of DTx 100, in accordance with various embodiments. At 202, access to DTx 100 may be provided. For example, DTx 100 made be made available as downloadable software for a local computing device, such as a desktop computer, laptop, tablet, smartphone, mobile device, or other computing devices. The local computing device may be associated with the patient or the patient's HCP. The local computing device may execute the software for rendering and processing therapeutic content 102 of DTx 100. Alternatively, DTx 100 may be hosted and executed on a network server and accessed by the local computing device over a wired or wireless network (such as via an API).

At 204, one or more memory task exercises 104 may be provided. For example, the software for DTx 100 may be executed on a network server or a local computing device. Execution of the software for DTx 100 may cause memory task exercises 104 to be provided to the patient via their computing device (such as mobile device). The local computing device may then render or display the memory task exercises 104. Alternatively, DTx 100 may be stored on the local computing device such that access to a network connection is not required to receive therapeutic content 102. The software for DTx 100 may include computer program instructions that, when executed by the computing device of the patient, effectuate one or more memory task exercises from memory task exercises 104. In some embodiments, the software for DTx 100 may include logic configured to determine a status of the patient during a treatment schedule, and may select the particular memory task exercises to be provided to the patient. For example, the software for DTx 100 may determine the memory task exercise based on a previously provided memory task exercise. In some embodiments, the software for DTx 100 may restrict access to memory task exercises that are not to be provided to the patient. For example, if there are ten memory task exercises in total, for a given treatment date/time, one of the memory task exercises may be selected and the remaining nine memory task exercises may be prevented from being accessed or otherwise provided to the patient.

At 206, one or more patient inputs associated with memory task exercises 104 may be received and processed. In some embodiments, a patient may provide an input via the patient's computing device. As an example, the patient may select a graphical user interface (GUI) rendered on a display screen of the patient's computing device. The selection may be detected via a touchscreen, via voice input, via eye tracking, or via other detection techniques, or combinations thereof. Furthermore, a patient may select an option using an input device coupled to the patient's computing device (such as using a computer mouse, a joystick, a wearable device, and the like). Upon detection of the input, such as detecting a change in capacitance at a particular location on a touchscreen indicative of a user touching that location on the touch screen, the computing device may determine an action, if any, to be performed in response. The action may be an executable action to be performed by the computing device, such as causing content to be rendered. Alternatively, the action may cause a message/request (such as an HTML) request to be sent from the computing device to another computing device, a server, or another computing component, or a combination thereof. For example, the patient inputs may be transformed into data requests transmitted across a network to a network server. Upon receiving the data requests, the network server may store data, retrieve data, send data to the computing device and/or another computing device, or perform other actions. As an example, the network server may select a memory task exercise and/or a psychotherapy lesson based on the submitted request, and may provide data including computer program instructions that, when executed by the local computing device of the patient, cause the memory task exercise and/or psychotherapy lesson to be rendered for the patient.

At 208, one or more of psychotherapy lessons 106 may be rendered. The particular psychotherapy lessons to be rendered at the local computing device may be selected based on a treatment schedule, a request from a patient and/or HCP of the patient, or other criteria, or combinations thereof. For example, the treatment schedule may indicate that at a first time (such as a first day of treatment), a first psychotherapy lesson is to be selected and data including the first psychotherapy lesson (such as computer programs designed to render particular content for the patient) may be provided to the patient's computing device, whereas, at a second time (such as an N-th day of treatment), a second psychotherapy lesson may be selected and provided to the patient's computing device. In some embodiments, the software for DTx 100 may be executed on a network server or a local computing device.

At 210, one or more messages may be generated and presented to the patient. The messages may be generated at a network server or on a local computing device. The messages may then be delivered and/or caused to be displayed, using a graphical user interface of the local computing device. In some cases, the messages may be dynamically generated using a chatbot, natural language processing technical, or other techniques. For instance, based on input parameters for a chatbot, which may be trained to generate messages having various input parameters, the chatbot may generate, and provided to the patient, a message used for messaging 108 of DTx 100. If messages are pre-generated and stored in memory, then a particular message may be selected based on input parameters associated with the psychotherapy lesson rendered at 208, feedback from the patient, instructions provided by the HCP, or based on other criteria, or combinations thereof. The stored messages may include labels indicating an emotion, concept, thought, phrase, mantra, or other cognitive message, conveyed by the respective messages. Based on the input parameters included within the request for the message (such as which may be generated after it is determined that the psychotherapy lesson has been consumed by the patient), a most appropriate message may be identified from the stored messages, and that message may be provided to the patient. For example, a similarity score may be computed based on a feature vector representing the message and a feature vector representing the request for a message (such as a Euclidean distance between a location of the requested message in a feature space and a location of each stored message may be computed).

In some embodiments, the rendering and/or processing of memory task exercises, psychotherapy lessons, and/or messaging may be done in order (such as first the memory task exercise, then the psychotherapy lesson, then the message). However, in some embodiments, the memory task exercises, psychotherapy lessons, and messaging may be performed in a different order (such as first a memory task exercise, then a psychotherapy lesson, then a message, then another message). Other orders of consuming DTx 100 are also possible.

In some embodiments, the delivery of memory task exercises 104, psychotherapy lessons 106, and/or messaging 108 may follow a predetermined treatment schedule that may be prescribed by an HCP or recommended by the provider of DTx 100. The delivery of therapeutic content 102 of DTx 100 according to the treatment schedule may be clinically validated for the treatment of one or more symptoms of depression. It will be appreciated that aspects of the treatment schedule may be adjusted or changed based on recommendations by the HCP and/or the specific circumstances of the patient utilizing DTx 100. For instance, in an example, the treatment schedule may have a six-week duration. As another example, the treatment schedule may have a four-week duration. The specific duration of the treatment schedule may be configured by the HCP or provider of DTx 100, and may be based on clinically supported evidence indicating an efficacy of the treatment to be provided.

### Emotional Face Memory Tasks (EFMT)

In some embodiments, memory task exercises 104 of DTx 100 may include emotional face memory task (EFMT) exercises. In some cases, the DTx 100 is implemented via computer program instructions such that, when executed, the computer program instructions cause one or more memory task exercises from memory task exercises 104 to be conveyed to the patient via the patient's computing device. The EFMT exercises may be configured to repair neural connectivity in disrupted brain circuitry via simultaneous emotion recognition and working memory tasks. The EFMT exercises can simultaneously engage two portions of the brain that regulate cognitive functions that can be impaired in patients experiencing MDD. The exercises may elicit activity in the patient's amygdala (such as which regulates emotion) and/or dorsolateral prefrontal cortex (such as which control cognition), leading to changes in short-term plasticity of the patient's brain networks. Accordingly, the EFMT exercises may address cognitive and emotional deficits often associated with MDD. Using N-back memory tasks (such as identifying an emotion of a person based on an image of the person's face while that person is emoting a particular emotion), the EFMT exercises aims to enhance cognitive control for emotional information processing.

During an EFMT exercise, a series of expression images may be sequentially displayed to the patient. An expression image refers to an image depicting a particular emotion. For example, an image may depict a human face expressing an emotion (such as anger, disgust, fear, and happiness). In some embodiments, each of the images may be displayed for a predetermined amount of time. The predetermined amount of time may include any suitable interval, such as between 0.1 and 5.0 seconds. Each of the images may be displayed for the same predetermined amount of time, however some images may be displayed for different amounts of time. In some cases, the images may be displayed for either a first predetermined amount of time or a second predetermined amount of time (or for other predetermined amounts of time). In some embodiments, the amount of time with which some or all of the images are displayed is random and/or configurable. For example, an HCP may determine that an amount of time with which an image is being displayed to the patient is too short or too long, and may adjust the amount of time. The adjustment may be made via the HCP inputting a request to their computing device, which sends an instruction to the patient's computing device to adjust the amount of time.

Each of the expression images may represent a respective expression within a set of expressions. The set of expressions may include any number of different emotions, such as happy, worried, angry, sad, surprised, disgusted, and the like. The expression images may be facial images or face images, which may each depict a face of a person depicting a particular emotion. The emotions depicted in the series of facial images may be displayed randomly or in a predetermined order. For example, such as an image depicting the emotion “happiness” may be followed by an image depicting the emotion “worry,” which may be followed by an image depicting the emotion “anger,” which may be followed by an image depicting the emotion “sadness,” which may be followed by an image depicting the emotion “surprise,” which may be followed by an image depicting the emotion “disgust.” The order of the depicted expression images may vary or repeat over the course of the EFMT exercise and/or across difference instances of the EFMT exercises. The predetermined order may be configured to trigger or engage certain areas of the patient's brain and/or to release one or more chemicals (such as acetylcholine, dopamine, norepinephrine, glutamate, serotonin, GABA, glycine, aspartate, epinephrine, nitric oxide and neuropeptide) to relieve symptoms of MDD.

The expression images may depict facial expressions of humans of any age, gender, and/or ethnicity. The expression images may be rendered in grey scale, color, or a combination thereof. The expression images may be in the form of photographs, illustrations, or animations. Persons of ordinary skill in the art will recognize that videos, gifs, audio, and/or other forms of content may be used instead of, or in addition to, the aforementioned image types. The expression images may be configured to correspond to a certain intensity of emotion (such as 90% intensity, 80% intensity, 70% intensity, 60% intensity, 50% intensity, etc.). It will be appreciated that expression images with higher intensity may be easier to identify than images with lower intensity levels. The intensity of the emotion of a given expression image maybe determined in advance by the HCP or other clinical support. For example, the depicted images may be assigned intensity “grades” by patients which may be used to compute an overall intensity of the emotion of the particular expression image. In some cases, the insanity of the emotion of a given expression image may be determined by using one or more computer vision techniques which compares the images to predetermined images classified as being of a particular intensity level. An embedding of the given expression image may be computed using the computer vision model, and, accordingly, a similarity between the given expression image's location in an n-dimensional features space of the computer vision model and a location in the feature space corresponding to a particular intensity of an emotion may be computed.

Each EFMT exercise may be configured as a modified N-back working memory task. That is, after a predetermined number of expression images are displayed, the patient may be asked whether the emotion observed on the currently presented expression image is the same as, or different than, the emotion observed on the image displayed N images prior (such as 1-back, 2-back, 3-back, 4-back, etc.). N may be any integer, such as 1, 2, 3, 4, 5, 6, 7, 8, 9, or greater. In some embodiments, the computer program instructions may cause a GUI to be rendered that allows the patient to provide an input/response via their computing device. After providing a response, one or more additional expression images may be displayed and the patient may again be asked whether the emotion observed on the currently presented image is the same as or different from the emotion observed on the image displayed N images back. In some embodiments, the next image displayed may be selected from a set of possible expression images based on the response provided by the patient. The response provided may be a binary response (such as yes or no). In some embodiments, a third option (such as “not sure” or “don't know”) may also be provided. Each response from the patient may be (a part of) a trial. The patient may be asked to complete any suitable number of trials during a round. For example, the number of trails in a round may be 1 or more, 5 or more, 10 or more, 15 or more, 20 or more, 25 or more, or other quantities of trails. As an illustrative example, a round may include 15, 16, 17, 18, 19, 20, 21, 22, 23, 24, or 25 trials. Each EFMT exercise may include any suitable number of rounds. For example, the number of rounds in a given EFMT exercise may be 1 or more, 5 or more, 10 or more, 15 or more, 20 or more, 25 or more, or other quantities of rounds. As an illustrative example, an EFMT exercise may include 10, 11, 12, 13, 14, 15, 16, 17, 18, 19, or 20 rounds.

In some embodiments, statistics regarding the responses provided by the patient may be obtained. The statistics may be used to determine an effectiveness of the EFMT exercise, an engagement level of the patient, or other aspects of the therapy. For example, in addition to providing an indication of the response, an amount of time between when an image was rendered and when a response was detected may be provided. Delay times that are deemed long (such as greater than a threshold amount of time, such as 2 seconds, 5 seconds, 10 seconds, 30 seconds, or other amounts of time) may indicate that emotion expressed by the rendered image may be unclear, indicating that the image may need to be removed from the EFMT exercise, updated, or otherwise adjusted to improve the image's therapeutic effectiveness.

The number of correct responses by the patient may be used to determine one or more scores. The scores may reflect the number of correct responses by the patient after completing some or all of the trials within a round or some or all of the rounds within an exercise. During the exercise, the patient's score may change or adjust with each new correct (or incorrect) response. The patient's overall score may reflect the number of correct responses submitted over the course of the exercise, at the end of the exercise and/or at the end of a set of exercises. In some embodiments, in addition to storing whether the patient was correct or incorrect, the response may indicate the response provided by the patient. The responses for a given EFMT exercise may be analyzed to determine response patterns, anomalies, or other aspects of the response, which can be used for improving the therapeutic treatment. For example, a number of instances with which the patient responded with a particular emotion (such as “sadness,” “depressed,” “anger,” etc.) may indicate the patient's mental state. Therefore, appropriate therapeutic interventions may be identified by the HCP and provided to the patient.

It will be appreciated that the difficulty level for successfully identifying whether the emotion observed on the currently presented image is the same as, or different than, the emotion observed on the image displayed N images prior may increase as the value of N increases. Conversely, the difficulty level may decrease as the value of N decreases. Accordingly, the value of N may be adjusted (either increased or decreased) to allow the patient to advance through the EFMT exercise, or a series of EFMT exercises, while remaining challenged at a level that is appropriate for the patient. The adjustment of integer N may be based at least in part on the percentage of correct responses from the total number of responses submitted. For example, N may be reset to N+1 if the percentage of correct responses is greater than a first threshold percentage at a predetermined time or reset to N−1 if the percentage of correct responses is less than a second threshold percentage at the predetermined time. N may not be adjusted if the percentage of correct responses is between the first and second threshold percentages. While N is adjusted by +1 or −1 in the above example, any adjustment factor may be used (such as 2, 3, 4, etc.). In some embodiments, the HCP may facilitate the adjustment of the integer N based on the results, monitored in real-time, may submitting an instruction via the HCP's computing device to adjust the integer N. In some embodiments, the integer N may be adjusted dynamically by DTx 100. For example, DTx 100 may include real-time monitoring of the difficulty of the treatment and may cause the integer N to be increased (or decreased) depending on the percentage of correct responses obtained from the patient.

Alternatively, or in addition to, adjusting the value of integer N, the difficulty level for successfully identifying whether the emotion observed on the currently presented image is the same as, or different than, the emotion observed on the image displayed N images prior may be increased or decreased by changing the intensity of emotion depicted in the expression images. For example, it may be more difficult for a patient to identify a happy emotion represented at a 50% intensity level versus a 90% intensity level because the lower intensity may provide less certainty as to the emotion being depicted. Accordingly, to make the exercise more challenging, one or more of the depicted expression images may be configured with a lower intensity level. Conversely, to the make exercise easier, one or more of the depicted expression images may be configured with a higher intensity level.

In some embodiments, the different images may be weighted. For instance, image data representing the images may include weights corresponding to a difficultly level of the images. The different weights may be selected such that images depicting an emotion with a higher intensity level are down-weighted while images depicting an emotion with a lower intensity are upweighted. Weighting the images based on emotional intensity level may improve the overall results by accounting for mistakes made by the patient for images that are more difficult to properly identify.

The integer N and/or emotion intensity level may be adjusted based on the patient's score at any suitable interval or stage of the EFMT exercise. For example, the integer N and/or emotion intensity level may be adjusted after the patient completes a portion of the trials within a round such that the level of difficulty changes as the patient proceeds within a round. The integer N and/or emotion intensity level may be adjusted after the patient completes all of the trials in the round such that the level of difficulty changes as the patient moves to the next round. The integer N and/or emotion intensity level may be adjusted after the patient completes all rounds of the exercise such that the level of difficulty changes as the patient moves to the next exercise. In some embodiments, the HCP may adjust the integer N and/or the emotion intensity level. In some embodiments, the integer N and/or the emotion intensity level may be automatically adjusted by DTx 100 based on the responses provided by the patient during a trail, round, and/or memory task exercise.

Further examples of EFMT exercises are described in U.S. Pat. No. 10,123,737 (issued Nov. 13, 2018) and U.S. Pat. No. 10,898,131 (issued Jan. 26, 2021), PCT Application No. PCT/US15/51791, filed on Sep. 23, 2015; U.S. patent application Ser. No. 17/156,195, filed on Jan. 22, 2021; and U.S. Provisional Patent Application Serial No. 62/054,371 filed on Sep. 23. 2014, which are each incorporated herein by reference in their entireties for all purposes.

### Cognitive Behavioral Therapy (CBT)

In some embodiment, psychotherapy lessons 106 of DTx 100 may include cognitive behavioral therapy (CBT) lessons. The CBT lessons may be configured to address other aspects of MDD that may not be specifically targeted by EFMT exercises, such as impairments in behavioral and social functioning that can exacerbate depressed mood. Each of the CBT lessons may include a video, such as an animation video or a partially animated video, a set of images, audio, haptic feedback, or other content, or combinations thereof. The CBT lessons may have a predetermined length (such as approximately 3-5 minutes). The specific length of the CBT lessons may be configured to facilitate ease of electronic storage and transmission of CBT lesson files. The specific length may also be configured to deliver the desired therapeutic effect in a duration that is more likely to maintain the patient's attention. The videos within the CBT lessons may be followed by a corresponding activity or task to be performed by the patient. Accordingly, the lessons may be configured to be internalized by the patient and acted upon.

The length of each CBT lesson may be carefully configured to maximize effectiveness, receptiveness, and impact. Longer lessons may be more difficult for certain patients to view in totality than shorter lessons. However, a lesson that is too short may not be effective in addressing the impairments in behavioral and social functioning associated with MDD. In some embodiments, a lesson's length may be adjusted (such as extended, shortened) so as to improve the effectiveness of the lesson's content.

Each of psychotherapy lessons 106 may have one or more parameters that are configurable to maximize an effectiveness and impact of the corresponding lesson. The parameters may include, but are not limited to (which is not to imply that other lists are limiting), a length of the psychotherapy lesson, a type of content of the psychotherapy lesson, and/or characteristics of the content. As mentioned above, the length of the psychotherapy lesson may be selected to maximize a likelihood that the patient receiving the content will consume the entirety of the psychotherapy lesson and retain the principles of the psychotherapy lesson. As an example, the psychotherapy lessons may be created to be within 2-3 minutes in duration. The type of content refers to a format with which the psychotherapy lesson may be conveyed. For example, the type of content may include an animated video, a partially animated video, a non-animated video, an image or set of images, textual content, audio, haptic feedback, or other content types, or combinations thereof. The characteristics of the content of the psychotherapy lesson may include a color spectrum used for the content (such as black and white, color, subset of colors), an effect applied to the content (such as blurring, rotations, edge smoothing, etc.), a speed with which characters/objects depicted within a video move (such as slowing down or speeding up characters/objects), whether the content is displayed as 2D video/image content or 3D video/image content, a complexity of the content (such as a grade level of the words used in the content), a narrative/concept of the content (such as if the video/audio/text depicts a story, selecting whether the story is a journey from one emotional state to another emotional state, or another story type), or other characteristics, or combinations thereof. As an example, certain words/phrases/images may be restricted from being shown to certain patients so as to not worsen a current emotional state of that patient. Some words/phrases/images may be restricted from use all together, or for a particular patient (such as providing children with suitable imagery/language which may or may not be used for adults). As another example, certain words/phrases/images may be pre-approved as being appropriate for use in some or all patients. As yet another example, if the psychotherapy lesson includes a video where a character moves (such as walks), the speed with which the character moves may be controlled to convey the appropriate emotional state to the patient. For instance, a character moving quickly can illicit anxious feelings in the patient, and so the psychotherapy lesson may be created such that the character's movement is moderate.

In some embodiments, the parameters of the psychotherapy lessons may be adjusted over a duration of the treatment schedule. For example, as the patient progresses through the treatment of DTx 100, the content may be configurable to account for improvements made by the patient or lack thereof. The content included in the psychotherapy lessons may be configured such that the objectives defining each psychotherapy lesson change over the duration of the treatment schedule. For example, the content included in a first psychotherapy lesson of the treatment schedule of a patient may be configured to introduce the patient to the treatment and bond/connect with the patient. In some cases, psychotherapy lessons 106 may include one or more “anchor” lessons. Within the treatment schedule, indications of when an anchor lesson is to occur may be stored. For example, the treatment schedule may indicate that every 5^{th} lesson is to be an anchor lesson, however the frequency with which the anchor lessons occur may vary and may be adjustable. The anchor lessons may be desired to anchor a particular lesson/technique/concept for the patient. In some cases, the anchor lessons may be available for quick reference by the patient so that the patient may re-reference the anchor lesson when needed. In some embodiments, anchor lessons may be stored locally on a client's computing device, whereas other psychotherapy lessons may be accessed via a server. As an example, an anchor lesson may include a breathing exercise, and the treatment may include performing the breathing exercise every N days, where N may be 1 or more.

Each CBT lesson may focus on, or leverage, one or more psychotherapy principles, such as emotion regulation (ER), behavioral activation (BA), and cognitive restructuring (CR). The psychotherapy principles associated with a respective CBT lesson (such as ER, BA, and/or CR) may be further supported with target messages to be conveyed to the patient. The psychotherapy principles may target the cognitive and emotional processing networks of the brain to restore them to their appropriate functioning. For instance, the psychotherapy lessons may support new skills acquired from the lesson's content to repair dysfunctional neural circuitry. In conjunction with EFMT, psychotherapy lessons 106 can provide significant improvement in the treatment of various mood disorders, such as MDD, as EFMT 104 can repair dysfunctional neural circuitry while psychotherapy lessons 106 can strengthen the repaired neural circuitry (as well as the surrounding/associated neural circuitry). Thus, the treatment program can be analogous to the process of treating a dysfunctional physical condition (such as a knee injury, shoulder injury, back injury, etc.). The treatment may include a surgical intervention to “repair” the injured portion of the body—akin to the EFMT, which “repairs” the neural circuitry of the brain—that may be followed by physical therapy to strengthen the injured portion and/or surrounding portions of the body—akin to the psychotherapy lessons, which “strengthen” the neural circuitry and/or surrounding/associated neural circuitry of the brain. As will be described below in greater detail, an additional intervention step (such as messaging 108) may be included to aid in maintaining the treatment's effectiveness.

ER principles may refer to the patient's ability to modulate or control the influence an emotion, or to modulate the degree to which an emotion is experienced. In the context of MDD treatment, lessons that leverage ER principles may enable the patient to experience distressing emotions, such as sadness, anger, or hopelessness, without becoming consumed by these emotions and responding in a detrimental manner. ER strategies within the CBT lesson may include mindfulness for thoughts and feelings, tolerating difficult emotions, and self-soothing techniques. Accordingly, CBT lessons directed to ER may be configured to provide the patient with skills and strategies to tolerate and overcome difficult emotions and moments the patient may encounter.

CBT lessons employing ER principles may further be configured to deliver one or more different target messages reinforcing such principles. For example, the ER-based CBT lesson may be configured to convey a “new way forward,” which may help to set patient expectations and enhance the patient's motivation and commitment to the treatment. The ER-based CBT lesson may be configured to convey “being mindful,” which may introduce the patient to the concept of “mindfulness,” including mindful breathing exercises. The ER-based CBT lesson may be configured to convey “mindfulness for difficult moments,” which may help the patient with identifying difficult moments and attention switching between difficult internal phenomena and the patient's breathing. The ER-based CBT lesson may be configured to convey “handling difficult moments,” which may provide or reinforce strategies for the patient to deal with sudden or intense negative emotions. The ER-based CBT lesson may be configured to convey a “different action,” which may help the patient to identify and act against harmful behavioral impulses associated with difficult emotions. The ER-based CBT lesson may be configured to convey a “relax, repair, refuel” mindset, which may educate the patient on stress and relaxation responses and/or provide exercises for guided muscle relaxation (such as progressive muscle relaxation (PMR)), and/or mindful relaxation.

BA principles may be used as a specific therapeutic technique within one or more CBT lessons. In a depressive cycle, the patient may be responding to sad, depressed, hopeless, and/or anxious moods by avoiding and/or disengaging from healthy behaviors, such as activities of daily living, physical activity and exercise, and interpersonal activity. This may act to exacerbate the patient's negative mood state. BA-based CBT lessons may target these behavior patterns by providing the patient with “calls to action” configured to help the patient re-engage with healthy activities and behaviors. Accordingly, BA-based CBT lessons may provide the patient with skills to identify and break negative emotion-behavior loops that may be causing the patient to withdraw from physical and/or interpersonal activities.

CBT lessons employing BA principles may further be configured to deliver one or more different target messages reinforcing such principles. For example, the BA-based CBT lesson may be configured to convey a “get going again” message, which may introduce the patient to the BA model of depression and help the patient overcome a lack of inertia with small steps. The BA-based CBT lesson may be configured to convey a “meaningful activities” mentality, which may help the patient explore values and/or schedule activities in meaningful life areas. The BA-based CBT lesson may be configured to convey a concept of “sleeping well,” which may provide the patient with a range of strategies for overcoming sleep disturbances. The BA-based CBT lesson may be configured to convey a “breaking free from worry” mindset, which may normalize and provide strategies to overcome perseverative thinking that may exacerbate depression and anxiety. The BA-based CBT lesson may be configured to convey a concept of “overcoming avoidance,” which may normalize and provide strategies for the patient to incorporate small steps to overcome various types or areas of avoidance. The BA-based CBT lesson may be configured to convey a notion of “more meaningful activities,” which may prompt the patient to revisit previous BA-based CBT lessons to explore and schedule activities in other meaningful life areas.

CR principles may be used as another therapeutic technique within one or more of the CBT lessons. The patient may be experiencing “automatic thoughts” involving negative perceptions of self, the world and the future. CR-based CBT lessons may be configured to provide the patient with cognitive tools to identify and systematically evaluate these thoughts for accuracy and validity when they arise, and to challenge and reframe the thoughts to be less maladaptive. Accordingly, CR-based CBT lessons may provide the patients with skills to identify cognitive distortions and/or problematic thought patterns and to adapt thereto.

CBT lessons employing CR principles may further be configured to deliver one or more different target messages reinforcing such principles. For example, the CR-based CBT lesson may be configured to convey a “balancing your thinking” mindset, which may introduce the patient to the cognitive model of depression and cognitive restructuring. The CR-based CBT lesson may be configured to convey an “all or nothing thinking” concept, which may teach the patient to identify and challenge all or nothing thinking for more balanced thinking and emotions. The CR-based CBT lesson may be configured to convey an “overcoming self-criticism” mentality, which may help the patient to deal with self-critical thinking in the moment and/or practice a guided self-compassion exercise. The CR-based CBT lesson may be configured to convey an “acting against negative beliefs” mindset, which may provide the patients with skills to identify and act against harmful behavioral impulses associated with negative core beliefs. The CR-based CBT lesson may be configured to convey a “flexible thinking” mentality, which may convey strategies to overcome cognitive inflexibility, particularly with respect to pessimistic or negative cognitive biases. The CR-based CBT lesson may be configured to convey a “maintain your gains” concept, which may recap key strategies from previous CBT lessons and provide recommendations to the patient for maintaining the learnings.

Table 1 includes a summary of the above CBT lessons that may be incorporated into therapeutic content 102.

TABLE 1

| No. | Category | Title |
|---|---|---|
| 1 | Emotion Regulation | A New Way Forward |
| 2 | Emotion Regulation | Being Mindful |
| 3 | Emotion Regulation | Mindfulness for Difficult Moments |
| 4 | Behavioral Activation | Get Going Again |
| 5 | Behavioral Activation | Meaningful Activities |
| 6 | Behavioral Activation | Sleeping Well |
| 7 | Cognitive Restructuring | Balancing Your Thinking |
| 8 | Cognitive Restructuring | All or Nothing Thinking |
| 9 | Cognitive Restructuring | Overcoming Self-Criticism |
| 10 | Emotion Regulation | Handling Difficult Moments |
| 11 | Emotion Regulation | Different Action |
| 12 | Emotion Regulation | Relax, Repair, Refuel |
| 13 | Behavioral Activation | Breaking Free From Worry |
| 14 | Behavioral Activation | Overcoming Avoidance |
| 15 | Behavioral Activation | More Meaningful Activities |
| 16 | Cognitive Restructuring | Acting Against Negative Beliefs |
| 17 | Cognitive Restructuring | Flexible Thinking |
| 18 | Cognitive Restructuring | Maintain Your Gains |

One or more of the CBT lessons listed in Table 1 may be configured to treat or address one or more elements of depression as defined by the Montgomery-Asberg Depression Rating Scale (MADRS). MADRS is a diagnostic questionnaire employed by HCPs, including psychiatrists, to measure the severity of depressive episodes associated with mood disorders. MADRS may include 10 evaluation items, which may be used to rate core symptoms of depression. The 10 evaluation items may include apparent sadness, reported sadness, inner tension, reduced sleep, reduced appetite, concentration difficulties, lassitude, inability to feel, pessimistic thoughts, and suicidal thoughts. In some embodiments, other metrics may be used to identify and/or classify a severity of depressive episodes associated with mood disorders.

Each MADRS item may be rated using a pre-defined four-step scale (such as 0, 2, 4, and 6 points) and three intermediate steps (such as 1, 3, and 5). An item rated at 0, for example, may indicate little or no symptoms are present while a rating of 6 may be indicative of severe or extreme symptoms. Items rated at 2 or 4 may suggest a level of severity that is greater than none but less than extreme. The three intermediate steps may denote worsening symptoms. An overall MADRS score may range from 0 to 60. A higher MADRS score may generally indicate a higher severity of depression. Table 2 includes an exemplary mapping of the CBT lessons in Table 1 to each of the 10 MADRS evaluation items. That is, the patient's completion of one or more of the CBT lessons in column A may lead to a lower score for the respective MADRS item and, thus, decrease the patient's overall MADRS score. The CBT lessons in column B represent a subset of column A. The patient's completion of one or more of the CBT lessons in column B may have a more direct and immediate impact on the respective MADRS item.

TABLE 2

| | CBT Lesson No(s). | |
|---|---|---|
| MADRS Item | A | B |
| Apparent | 2, 3, 4, 5, 7, 8, 9, 10, 11, | 5, 8, 9, 10, 15, 16, 18 |
| Sadness | 13, 14, 15, 16, 17, 18 | |
| Reported | 2, 3, 4, 5, 7, 8, 9, 10, 11, | 5, 8, 9, 10, 15, 16, 18 |
| Sadness | 13, 14, 15, 16, 17, 18 | |
| Inner Tension | 2, 3, 7, 8, 9, 10, 12 | 2, 3, 10, 12 |
| Reduced Sleep | 4, 5, 6, 15 | 6 |
| Reduced | — | — |
| Appetite | | |
| Concentration | 2, 3 | — |
| Difficulties | | |
| Lassitude | 1, 4, 5, 11, 14, 15 | 4 |
| Inability to Feel | 2, 3, 4, 5, 6, 15 | — |
| Pessimistic | 2, 3, 7, 8, 10, 11, 13, 16, | 8, 17 |
| Thoughts | 17, 18 | |
| Suicidal | 2, 3, 9, 10, 11, 12, 16, 18 | — |
| Thoughts | | |

One or more of the CBT lessons listed in Table 1 may be configured to treat or address one or more elements of depression as defined by the Hamilton Depression Rating Scale (HDRS). HDRS is a clinician-administered depression assessment scale configured to measure the severity of symptoms of depression. HDRS may include 17 evaluation items (HAM-D17), such as depressed mood, feelings of guilt, suicide, early insomnia, middle insomnia, late insomnia, work and activities, retardation, agitation, psychic anxiety, somatic anxiety, somatic symptoms (GI), general somatic symptoms, genital symptoms, hypochondriasis, loss of weight, and/or insight.

Each of the items in HAM-D17 may be evaluated or scored on a predetermined scale (such as 0 to 2, 0 to 3, or 0 to 4). An item rated at 0, for example, may indicate little or no symptoms are present while the highest score may indicate the presence of severe or extreme symptoms. Items rated in between may suggest a level of severity that is greater than none but less than extreme. An overall HAM-D17 score may range from 0 to 53, with higher scores generally indicating a higher severity of depression. The level of severity may be assessed according to pre-defined ranges on the HAM-D17 scale. For example, an overall score of 0-7 may indicate no depression. An overall score of 8-13 may indicate a mild level of depression. An overall score of 14-18 may indicate a mild-to-moderate level of depression. An overall score of 19-22 may indicate a moderate-to-severe level of depression. An overall score of 23 or above may indicate a very severe level of depression. Table 3 includes an exemplary mapping of the CBT lessons in Table 1 to each of the 17 evaluation items in HAM-D17. That is, the patient's completion of one or more of the CBT lessons in column A may lead to a lower score for the respective HAM-D17 item and, thus, decrease the patient's overall HAM-D17 score. The CBT lessons in column B represent a subset of column A. The patient's completion of one or more of the CBT lessons in column B may have a more direct and immediate impact on the respective HAM-D17 item.

TABLE 3

| | CBT Lesson No(s). | |
|---|---|---|
| HDRS Item | A | B |
| Depressed | 2, 3, 4, 5, 7, 8, 9, 10, 11, | 5, 8, 9, 10, 15, |
| Mood | 13, 14, 15, 16, 17, 18 | 16, 18 |
| Feelings | 2, 3, 9, 10, 13 | 9, 13 |
| of Guilt | | |
| Suicide | 2, 3, 9, 10, 11, 12, 16, 18 | — |
| Insomnia, Early | 4, 5, 6, 15 | 6 |
| Insomnia, Middle | 4, 5, 6, 15 | 6 |
| Insomnia, Late | 4, 5, 6, 15 | 6 |
| Work and | 4, 5, 14, 15 | 5, 15 |
| Activities | | |
| Retardation | 1, 4, 5, 14, 15 | 4 |
| Agitation | 2, 3, 4, 5, 10, 12, 15 | 12 |
| Anxiety, Psychic | 2, 3, 7, 8, 10, 12, 13 | 10, 13 |
| Anxiety, Somatic | 2, 3, 10, 12 | 12 |
| Somatic | — | — |
| Symptoms, | | |
| GI | | |
| Somatic | 2, 3, 4, 5, 12, 15 | 12 |
| Symptoms, | | |
| General | | |
| Genital Symptoms | — | — |
| Hypochondriasis | 7, 8, 13 | — |
| Loss of Weight | | — |
| Insight | 1, 4, 7, 18 | — |

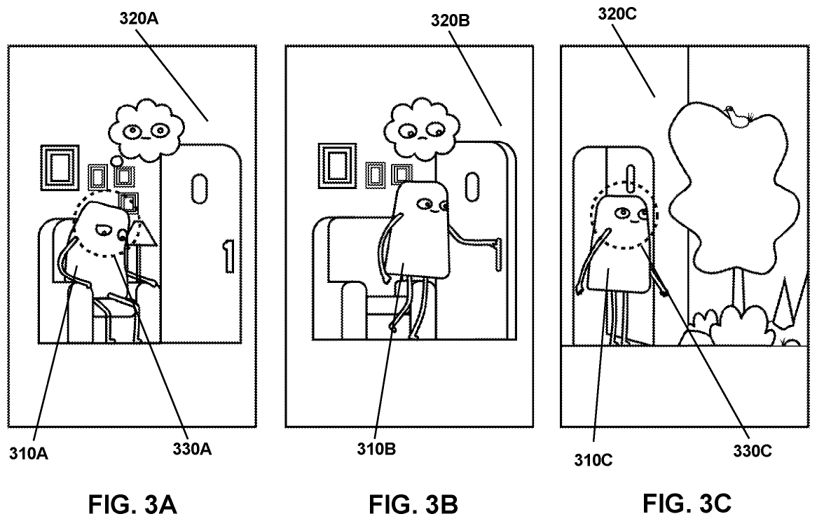

FIGS. 3A-3C, 4A, 4B, 5A, and 5B depict examples of visuals and/or features within therapeutic content 102 of one or more CBT lessons, in accordance with various embodiments. In some embodiments, as mentioned above, psychotherapy lessons 106 may include videos, such as animated videos. The visuals and features within therapeutic content 102 of a given CBT lesson may be displayed in the frames of an animated video. In some embodiments, psychotherapy lessons 106 may further include one or more tasks and/or exercises to be performed by the patient. The tasks and/or exercises may be provided prior to, during, or after the video, for instance. In some embodiments, the videos and/or therapeutic content may be provided to the patient via the patient's computing device. In some embodiments, the therapeutic content may be provided to the patient via a user interface of a mobile application. The mobile application may include computer program instructions to cause the therapeutic content to be rendered via the patient's computing device. In some cases, the therapeutic content may be stored locally in memory on the patient's computing device, however alternatively or additionally, therapeutic content may be accessed from a network server storing some or all of the prescribed therapeutic content.

By way of example, FIG. 3A depicts a first frame of a video of a psychotherapy lesson. The first frame may include an icon 310A, which may represent an entity (such as a character in a story) in a visual representation of a surrounding environment 320A. The entity represented by the icon 310A may be associated with symptoms of a mood disorder or mood state. In this example, the symptoms of the mood disorder or a mood state may be a state of sadness and/or a state of unhappiness, as reflected by an expression 330A of the entity depicted by icon 310A. As used herein, the term “icon” may refer to a visual representation of a seemingly living entity such as humanoid character or non-humanoid object. The entity may have moving features such as moving arms, moving legs, moving torso, moving facial features, or the like, when presented in the context of a video animation. The movements of the entity may be adjusted as the video progresses (such as from faster moving features to slower moving features). In some embodiments, characteristics of the entity may change over the duration of the video or over the duration of one or more videos. For example, a color scheme of the entity and/or scene may change from a beginning of a video to an end of the video.

In the example video depicted by FIGS. 3A-3C, the entity represented by the icon 310A is shown transitioning from the environment 320A in FIG. 3A to an environment 320B in FIG. 3B, and then to an environment 320C in FIG. 3C. For example, the entity depicted by the icon 310A in FIG. 3A has an expression 330A that may correspond to a sad mood state or an unhappy mood state. The entity represented by the icon 310A may change mood states (such as change from sad to happy) when transitioning from environment 320A to environment 320C. That is, expression 330A of the entity represented by the icon 310A in FIG. 3A may change to expression 330C in FIG. 3C. In this example, environment 320A of FIG. 3A may be dark and indoors. Environment 320C of FIG. 3C, however, may be bright and outdoors. The entity depicted by icons 310A, 310B, 310C may transition from environment 320A to environment 320C by using an exit door depicted within environment 320B. The transition from environment 320A to environment 320C may occur during a predetermined amount of time (such as 5 seconds, 7 seconds, 10 seconds, etc.), which may be configured to mitigate one or more symptoms of MDD. For example, an amount of time with which the transition from environment 320A to 320C occurs may be increased or decreased based on the particular therapy being provided to the patient, the patient's progression through the therapy, feedback from the patient (such as explicit feedback, such as responses to questions/prompts, and/or implicit feedback captured via one or more sensors, such as biometric feedback, eye tracking, etc.). However, the present disclosure is not limited to these specific environments or this specific transition between environments 320A-320C and each can be visually presented in other ways to achieve the same meaning being conveyed by the entity's transition from environments 320A to 320C.

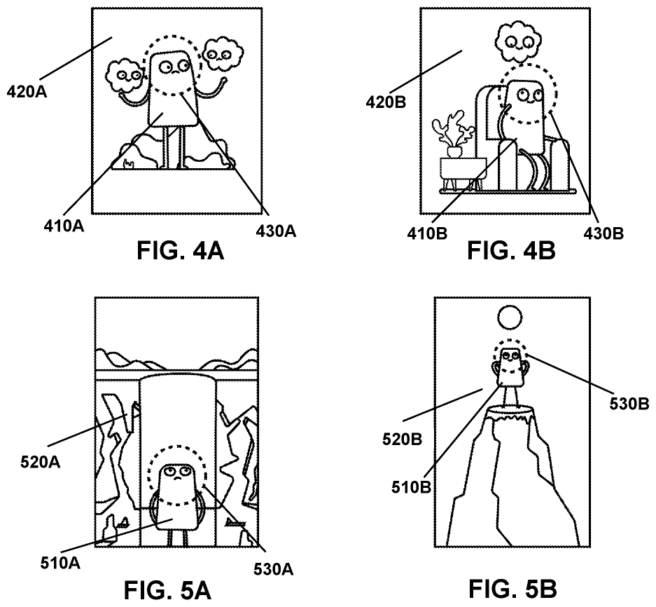

Other examples of an entity transitioning between environments and mood states are shown in FIGS. 4A, 4B, 5A, and 5B. For example, FIG. 4A depicts a frame from a video of a psychotherapy lesson, and may include an icon 410A representing an entity expressing an expression 430A (such as unhappiness or depression) in an environment 420A. Upon transitioning to an environment 420B, as shown in a frame from the video of the psychotherapy lesson of FIG. 4B, an icon 410B representing the entity transitions to an expression 430B (such as happiness). Similarly, FIG. 5A depicts a frame from a video of a psychotherapy lesson, and may include an icon 510A representing an entity expressing an expression 530A (such as unhappiness or depression) in an environment 520A (such as in a cave below ground). Upon transitioning to an environment 520B (such as on a mountain that rose from below ground in the environment 520A to above ground in 520B), as shown in a frame from the video of the psychotherapy lesson of FIG. 5B, an icon 510B representing the entity and transitions to expression 530B (such as happiness, confidence, etc.). Each of these respective transitions may be configured to occur over predetermined time periods and/or have accompanying audio effects, which may help to alleviate one or more symptoms of MDD.

Each of the exemplary frames depicted in FIGS. 3A-3C, 4A, 4B, 5A, and 5B may be, individually or together with one or more other frames, part of an animation video that may be displayed during a CBT lesson. The animation video may include text content, image content, video content, audio content, or any combination thereof. The animation video may graphically depict an entity moving between environments with associated changes in mental or emotional states. These transitions may help train the patient to use cognitive skills that may enable the patient to make similar emotional transitions in real life. The animation video and its accompanying features and graphics, when viewed by the patient as part of a treatment schedule associated with DTx 100, may trigger the release of one or more chemicals in the patient's brain (such as dopamine, serotonin, endorphins, etc.) that promote a healthier mental and/or emotional state.

### Treatment Schedule

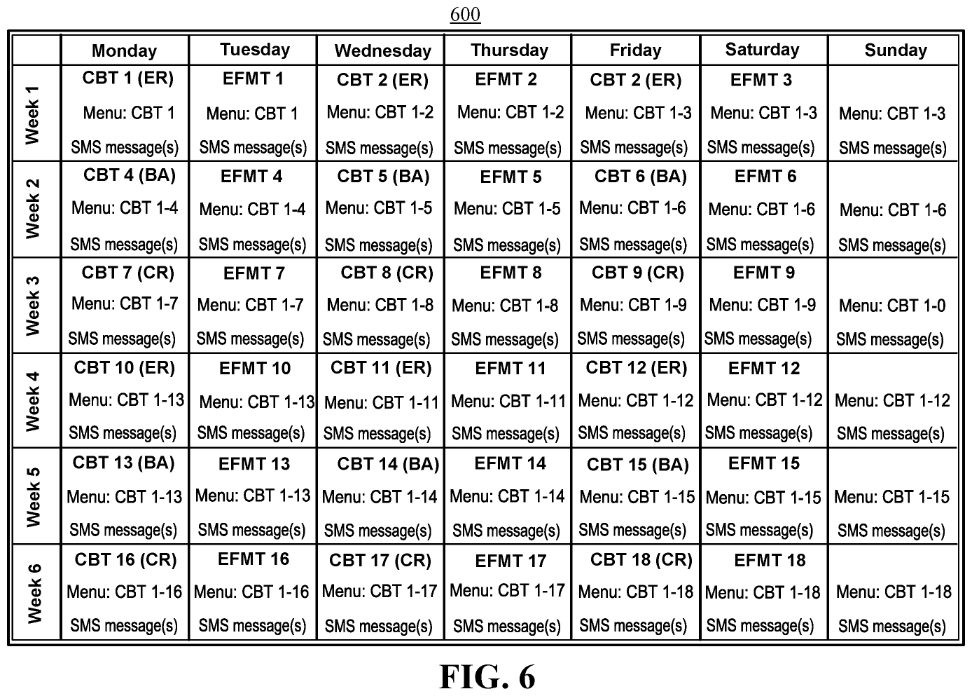

FIG. 6 depicts an exemplary treatment schedule 600 for delivering therapeutic content 102 via DTx 100. As shown in FIG. 6, treatment schedule 600 may include a six-week treatment period, though the period may be shortened or extended depending upon the patient's response to the treatment. Treatment schedule 600 may be prescribed by the patient's HCP or recommended by the provider of DTx 100. During each week of treatment schedule 600, the patient may complete three (3) memory task exercises (such as EFMT) and three (3) psychotherapy lessons (such as CBT). Accordingly, over the course of the six-week treatment period, the patient may complete 18 EFMT exercises and 18 CBT lessons. In addition, messages (such as SMS messages) may be delivered during each day of the six-week treatment period. Persons of ordinary skill in the art will recognize that the particulars of the treatment schedule may vary from patient to patient, and treatment schedule 600 represents one example. Different frequencies of EFMT exercises, CBT lessons, and/or messages may be implemented by a treatment schedule. In some embodiments, the HCP or provider of DTx 100 may generate the treatment schedule for the patient. In some embodiments, one or more pre-generated treatment schedules may be used as a patient's treatment schedule (such as using a generated treatment schedule for a patient having similar patient characteristics as another patient), or as a basis for creating a new treatment schedule for a patient (such as by modifying one or more aspects of an existing treatment schedule).

Treatment schedule 600 may be designed to deliver therapeutic content 102 to a patient via DTx 100. As shown in FIG. 6, treatment schedule 600 may include a six-week treatment period, though the period may be shortened or extended depending upon the patient's response to the treatment. Treatment schedule 600 may be prescribed by the patient's HCP or recommended by the provider of DTx 100. Furthermore, the treatment schedule may be adjusted as the therapeutic content is delivered to the patient. For example, one or more additional psychotherapy lessons may be added to a current treatment schedule, a frequency with which messages are provided to the patient may be increased or decreased, and the like. In some embodiments, parameters regarding the therapeutic content included within the treatment schedule may be adjusted during the treatment schedule's duration. For example, a color scheme, word list, content type, character motion speed, and the like, may be adjusted based on the HCP's input, feedback provided from the patient, performance metrics measuring an effectiveness of the therapy, or based on other measures, or combinations thereof.

During each week of treatment schedule 600, the patient may complete 3 memory task exercises (such as EFMT) and 3 psychotherapy lessons (such as CBT). Accordingly, at the conclusion of the six-week treatment period, the patient may complete 18 EFMT exercises and 18 CBT lessons. The 18 EFMT exercises are shown in FIG. 6 as EFMT 1, EFMT 2, EFMT 3, EFMT 4, EFMT 5, EFMT, EFMT 6, EFMT 7, EFMT 8, EFMT 9, EFMT 10, EFMT 11, EFMT 12, EFMT 13, EFMT 14, EFMT 15, EFMT 16, EFMT 17, and EFMT 18. The 18 CBT lessons are reflected in Table 1 above and are shown in FIG. 6 as CBT 1, CBT 2, CBT 3, CBT 4, CBT 5, CBT 6, CBT 7, CBT 8, CBT 9, CBT 10, CBT 11, CBT 12, CBT 13, CBT 14, CBT 15, CBT 16, CBT 17, and CBT 18. In addition to the EFMT exercises and CBT lessons, messages (such as SMS messages) may be delivered during each day of the six-week treatment period in treatment schedule 600.

Although not shown in FIG. 6, therapeutic content 102 may be provided by DTx 100 as an adjunct to other types of treatments, such as antidepressant therapies (ADTs), which may include serotonin reuptake inhibitors (SSRIs), serotonin and norepinephrine reuptake inhibitors (SNRIs) and norepinephrine and dopamine reuptake inhibitors (NDRIs). The ADTs may be prescribed by the patient's HCP in accordance with a separate treatment schedule.

As shown in the example of treatment schedule 600, the EFMT exercises and the CBT lessons may be completed on alternating days of the week. For example, the CBT lessons may be completed on Mondays, Wednesdays and Fridays and the EFMT exercises may be completed on Tuesdays, Thursdays and Saturdays, though it will be appreciated that therapeutic content 102 may be scheduled in any suitable order. For instance, the CBT lessons may be completed on Tuesdays, Thursdays, and Saturdays and the EFMT exercises may be completed on Mondays, Wednesdays, and Fridays. One or more of the EFMT exercises and CBT lessons may also be completed on the same day. In addition, while treatment schedule 600 depicted in FIG. 6 may not include any scheduled EFMT exercises or CBT lessons on Sundays, it will be further appreciated that such “off” days may fall on any day of the week (such as Monday to Saturday) while still permitting the EFMT exercises and the CBT lessons to be completed on alternating days of the week. For example, the CBT lessons may be completed on Tuesdays, Thursdays and Saturdays and the EFMT lessons may be completed on Wednesdays, Fridays and Sundays, leaving Mondays as the “off” day. In other embodiments, the “off” day may vary from week-to-week over the course of the six-week treatment period. Still further, some embodiments may include enabling a patient to access certain exercises and/or lessons at various points throughout the treatment schedule. For example, a patient may be able to access an anchor lesson and any point throughout treatment schedule 600 even if a different psychotherapy lesson or no psychotherapy lesson is scheduled to occur on that particular day.

Each of the exercises in EFMT 1-18 may have a predetermined duration (such as approximately 30 minutes). The expression images displayed in EFMT 1-18 may be the same or may vary from exercise-to-exercise. In addition, the emotion intensities of the expression images displayed in EFMT 1-18 may be the same or may vary from exercise-to-exercise. By varying the expression images and/or the emotion intensities of the displayed expression images, the difficulty level of the exercises in EFMT 1-18 may be increased or decreased during the six-week treatment period. For example, the exercises in EFMT 1-18 may get progressively more challenging (such as by decreasing the emotion intensities of the displayed expression images) as the patient progresses through treatment and is able to provide a predetermined number of correct responses. Conversely, the exercises in EFMT 1-18 may become less challenging (such as by increasing the emotion intensities of the displayed expression images) if the patient is not able to provide a predetermined number of correct responses during the exercises. In other embodiments, the difficulty level may vary (such as both increase and decrease) throughout treatment schedule 600 depending upon the patient's performance at any given time during the six-week treatment period.

To select the different images to be used in a given EFMT, the DTx 100 may filter the images based on intensity labels indicating an emotion intensity level of a corresponding image. For example, images labeled as being related to a first emotion (such as anger) and having less than a first emotion intensity level (such as less than an emotion intensity level of 3) may be identified from some or all available expression images. In some embodiments, a HCP may input a script, input values into database search fields, or use another selection mechanism to select the emotions to be included within a given EFMT and/or an emotion intensity level. The HCP may be returned with a listing of results satisfying the criteria of their search, and may select some or all of the images included within the results for creating an EFMT. In some embodiments, the EFMT creation process may be automated such that the filtering of the images and selection from the results are performed automatically. In this scenario, a HCP or other provider of DTx 100 may be able to subsequently modify and/or curate the selected images.

In addition, or alternatively, to varying the expression images and their associated emotion intensities, the difficulty level of the exercises in EFMT 1-18 may be adjusted by changing the number of N images back the patient may be asked to recall. That is, the difficulty level of the exercises in EFMT 1-18 may be increased by increasing the integer N and decreased by lowering the integer N. Similarly to the above techniques with which the emotions and/or emotion intensity levels of the images may be selected, the value of integer N may also be selected/modified by a HCP and/or a provider of DTx 100. As with adjustments to the emotion intensities of the displayed expression images, the integer N may be adjusted based on the patient's performance during treatment. For example, the integer N may be increased if the number of correct responses by patient exceeds a predetermined threshold or may be decreased if the number of correct responses is lower than the predetermined threshold. The integer N may be modified via a HCP inputting a value for integer N into a GUI on their computing device, which may generate and transmit an instruction to DTx 100 to set the new value for integer N. Accordingly, the integer N may be used to maintain a difficulty level that is best suited for the patient's abilities at a given time during treatment schedule 600.

Each of the lessons in CBT 1-18 may have a predetermined duration (such as approximately 3-5 minutes) and may include an animated video. The content of the lessons in CBT 1-18 may vary from lesson-to-lesson over the course of treatment schedule 600, although as further discussed below the patient may elect to, and/or the patient's HCP may recommend that, the patient retake a respective CBT lesson one or more times. While the content of the lessons in CBT 1-18 may vary, the lessons in CBT 1-18 may be grouped in subsets to provide therapeutic intervention according to certain psychotherapy principles, including emotion regulation (ER), behavioral activation (BA) and cognitive restructuring (CR). For example, as shown in Table 1 above and in FIG. 6, the lessons in CBT 1-3 and 10-12 may be grouped to provide therapeutic intervention through ER. The lessons in CBT 4-6 and 13-15 may be grouped to provide therapeutic intervention through BA. The lessons in CBT 7-9 and 16-18 may be grouped to provide therapeutic intervention through CR.

In addition to grouping the lessons in CBT 1-18 according to ER, BA and CR, treatment schedule 600 may be configured to deliver the lessons in CBT 1-18 in a respective sequence to optimize the manner in which the patient receives therapeutic content 102, thereby leading to better treatment outcomes. For example, as shown in FIG. 6, the lessons in CBT 1-3 (ER) may be completed by the patient during week 1. The lessons in CBT 4-6 (BA) may be completed during week 2. The lessons in CBT 7-9 (CR) may be completed during week 3. The lessons in CBT 10-12 (ER) may be completed during week 4. The lessons in CBT 13-16 (BA) may be completed during week 5. The lessons in CBT 16-18 (CR) may be completed during week 6. The particular sequence with which the CBT lessons are selected may relate to the particular EFMT exercises selected, with the purpose of the EFMT exercise repairing particular neural circuitry and the CBT lesson strengthening the repaired neural circuitry. Although the foregoing describes a particular configuration of treatment schedule 600, alternative arrangements of the psychotherapy lessons may be used.

Accordingly, treatment schedule 600 may be configured to introduce principles of ER to the patient during week 1, which may teach the patient how to experience distressing emotions, such as sadness, anger, or hopelessness, without becoming consumed by these emotions and without acting in a manner that is detrimental. The lessons in CBT 1-3 (ER) may provide the patient with strategies including mindfulness for thoughts and feelings, tolerating difficult emotions, and self-soothing techniques.

During week 2, treatment schedule 600 may be configured to introduce principles of BA. That is, the lessons in CBT 4-6 (BA) may target patterns in which the patient may be avoiding or disengaging from healthy activities or behaviors. The lessons in CBT 4-6 (BA) may provide the patient with tasks or “calls to action” designed to help the patient re-engage in such activities and behaviors.

During week 3, treatment schedule 600 may be configured to introduce principles of CR, which may target the patient's negative perceptions of self, the world and/or the future. Thus, the lessons in CBT 7-9 (CR) may provide the patient with cognitive tools to identify and systematically evaluate such negative thoughts for accuracy and validity. The lessons in CBT 7-9 (CR) may help the patient to challenge and reframe the thoughts to be less maladaptive.

During weeks 4-6 of treatment schedule 600, the sequence of ER, BA and CR lessons may repeat. For example, the lessons in CBT 10-12 (ER), the lessons in CBT 13-15 (BA) and the lessons in CBT 16-18 (CR) may be completed during weeks 4, 5, and 6, respectively. While the principles of ER, BA and CR may be repeated, the specific content of the lessons in CBT 10-12 (ER), CBT 13-15 (BA) and CBT 16-18 (CR) may be different from the content that was delivered during weeks 1-3. For example, as the patient progresses through weeks 1-3 of treatment schedule 600, the patient may become more adept at practicing the skills and principles associated with ER, BA and CR. Thus, during weeks 4-6, the lessons in CBT 10-12 (ER), CBT 13-15 (BA) and CBT 16-18 (CR) may be configured to provide more advanced strategies that reinforce previous learnings and/or convey additional (such as more sophisticated) mechanisms and techniques for alleviating the patient's symptoms of depression.

In addition to completing the exercises in EFMT 1-18 and/or the lessons in CBT 1-18, DTx 100 may be further configured to provide the patient with a menu option for selecting one or more of the lessons in CBT 1-18 over the course of treatment schedule 600. The menu option may be configured to allow the patient to retake a previously completed lesson in CBT 1-18, which may help to reinforce previous learnings and/or provide the patient with additional opportunities to master the strategies and techniques being taught. For example, after completing the lesson in CBT 1 on Monday of week 1, the patient may be able to retake the lesson in CBT 1 anytime over the remainder of treatment schedule 600. However, the menu option may not permit the patient to take other lessons until the lessons have been completed in accordance with treatment schedule 600. Thus, the menu option may not provide an opportunity for the patient to access the lesson in CBT 9, for example, until that lesson has been completed on Thursday of week 3. In some embodiments, the menu option may be presented to the patient via a UI rendered via the patient's computing device.

As part of treatment schedule 600, messages may be generated and delivered to the patient on a periodic basis. Messages may be selected to reinforce the principles from the lessons in CBT 1-18 and may be synchronized with the patient's progress. Messaging 108 may facilitate the delivery of messages to the patient at any desired intervals (such as 0-4 messages per day). The messages may also include reminders for the patient to complete the exercises in EFMT 1-18 and/or the lessons in CBT 1-18. The messages may also be configured to acknowledge the difficulty, or utility, of completing the exercises in EFMT 1-18 and/or the lessons in CBT 1-18 to facilitate patient engagement and motivation. Messaging 108 may be personalized based on the patient's activity, adherence, and/or performance over the course of treatment schedule 600. In some embodiments, the messages may be rendered within a UI of a mobile application executing on the patient's computing device. The messages may be displayed when the patient invokes the mobile application (such as DTx 100), as well as, or alternatively, at particular time intervals. For example, the messages may be displayed at a particular time or times every day. In some cases, notifications may be displayed (such as “pop ups”) that reflect some or all of the message or a snippet/synopsis of the message.

### Exemplary Systems and Computing Devices

FIG. 7 is a diagram of a system 700 for treating depression via DTx 100, in accordance with various embodiments. System 700 may include a user device 710, an application server 720, and a network 730. User device 710 may submit a request 712 for therapeutic content 102 from application server 720. Request 712 may include, for example, an indication of a user of user device 710 accessing a website or web portal hosted by the application server 210 (or other server) and requesting to download the therapeutic content to the user device 710. For example, request 712 may include a MAC identifier, IP address, user/patient identifier, or other identifier used to identify the patient (such as an operator of user device 710). As described herein, user device 710 is referred to interchangeably as a “computing device.” User device 710 may receive a message such as an SMS message or email message with a hyperlink referencing a network location for application server 720 storing therapeutic content 102. User device 710 may receive a selection of the hyperlink from the user. Responsive to the selection of the hyperlink, user device 710 may transmit request 712 for downloading therapeutic content 102 via network 730.

Responsive to the request 712, the application server 720 may provide data 722, which may include therapeutic content 102. Upon receipt of the data 722, the user device 710 may render or display therapeutic content 102 in accordance with a treatment schedule hat is configured to alleviate one or more MDD symptoms the user may be experiencing. In other embodiments, therapeutic content 102 may be rendered from the application server 720 to the user device 710 via the network 730.

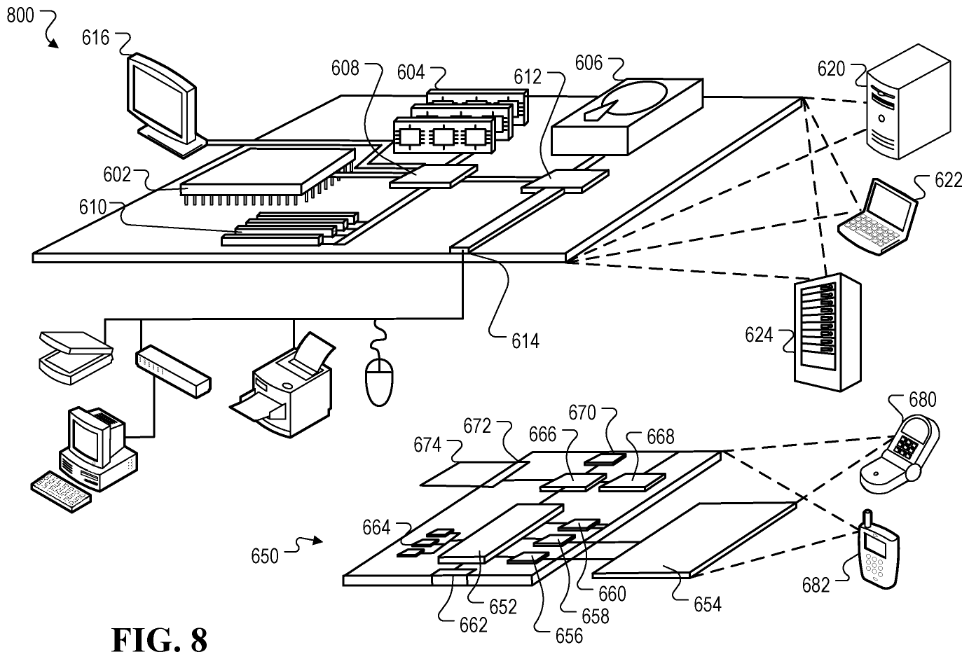

FIG. 8 is a diagram of exemplary system components of devices for treating depression via DTx 100, in accordance with various embodiments. Computing device 800 is intended to represent various forms of digital computers, such as laptops, desktops, workstations, personal digital assistants, servers, blade servers, mainframes, and other appropriate computers. Computing device 650 is intended to represent various forms of mobile devices, such as personal digital assistants, cellular telephones, smartphones, and other similar computing devices. Additionally, computing device 800 or 650 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be examples only, and are not meant to limit implementations of the inventions described and/or claimed in this document.

Computing device 800 includes a processor 602, memory 604, a storage device 606, a high-speed interface 608 connecting to memory 604 and high-speed expansion ports 610, and a low-speed interface 612 connecting to low-speed expansion port 614, and storage device 606. Each of components 602, 604, 606, 608, 610, and 612, are interconnected using various busses, and can be mounted on a common motherboard or in other manners as appropriate.

Processor 602 can process instructions for execution within computing device 800, including instructions stored in memory 604 or on storage device 606 to display graphical information for a GUI on an external input/output device, such as display 616 coupled to high-speed interface 608. In other implementations, multiple processors and/or multiple buses can be used, as appropriate, along with multiple memories and types of memory. Also, multiple instances of computing devices 800 can be connected, with each device providing portions of the necessary operations, such as a server bank, a group of blade servers, or a multi-processor system. Additionally, processors 652 may include multiple instances of a processor or set of processors.

Memory 604 may store information within computing device 800. In some embodiments, memory 604 is a volatile memory unit or units. In another implementation, memory 604 is a non-volatile memory unit or units. Memory 604 can also be another form of computer-readable medium, such as a magnetic or optical disk.

Storage device 606 is capable of providing mass storage for computing device 800. In some embodiments, storage device 606 can be or contain a computer-readable medium, such as a floppy disk device, a hard disk device, an optical disk device, or a tape device, a flash memory or other similar solid-state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as memory 604, storage device 606, or memory on processor 602.

High-speed interface 608 can manage bandwidth-intensive operations for computing device 800, while low-speed interface 612 can manage lower bandwidth intensive operations. Such allocation of functions is only an example. In some embodiments, high-speed interface 608 may be coupled to memory 604, display 616, such as through a graphics processor or accelerator, and to high-speed expansion ports 610, which can accept various expansion cards (not shown). In some embodiments, low-speed interface 612 may be coupled to storage device 606 and low-speed expansion port 614. Low-speed expansion port 614, which can include various communication ports, such as USB, Bluetooth, Ethernet, and/or wireless Ethernet, can be coupled to one or more input/output devices, such as a keyboard, a pointing device, a microphone/speaker pair, a scanner, and/or a networking device such as a switch or router, such as through a network adapter. Computing device 800 can be implemented in a number of different forms, as shown in the figure. For example, computing device 800 can be implemented as a standard server 620, or multiple times in a group of such servers. Computing device 800 can also be implemented as part of a rack application server 624. In addition, computing device 800 can be implemented in a personal computer such as a laptop computer 622. Alternatively, components from computing device 800 can be combined with other components in a mobile device (not shown), such as device 650. Each of such devices can contain one or more of computing device 800, 650, and an entire system can be made up of multiple computing devices 800, 650 communicating with each other.

Computing device 800 can be implemented in a number of different forms, as shown in the figure. For example, computing device 800 can be implemented as a standard server 620, or multiple times in a group of such servers. Computing device 800 can also be implemented as part of a rack application server 624. In addition, computing device 800 can be implemented in a personal computer such as a laptop computer 622. Alternatively, components from computing device 800 can be combined with other components in a mobile device (not shown), such as device 650. Each of such devices can contain one or more of computing device 800, 650, and an entire system can be made up of multiple computing devices 800, 650 communicating with each other.

Computing device 650 may include a processor 652, memory 664, and an input/output device such as a display 654, a communication interface 666, and a transceiver 668, among other components. Computing device 650 can also be provided with a storage device, such as a micro-drive or other device, to provide additional storage. Each of components 650, 652, 664, 654, 666, and 668, may be interconnected using various buses, and several of the components can be mounted on a common motherboard or in other manners as appropriate. As described herein, a single instances of certain components of computing devices 800, 650 may be depicted within FIG. 8, however it should be understood that additional instances of those components may be implemented and the use of a single instance of the components is only to ensure that the components are not obfuscated.

Processor 652 can execute instructions within computing device 650, including instructions stored in memory 664. Processor 652 can be implemented as a chipset of chips that include separate and multiple analog and digital processors. Additionally, processor 652 can be implemented using any of a number of architectures. For example, processor 652 can be a CISC (Complex Instruction Set Computers) processor, a RISC (Reduced Instruction Set Computer) processor, or a MISC (Minimal Instruction Set Computer) processor. Processor 652 can provide, for example, for coordination of the other components of the device 650, such as control of user interfaces, applications run by device 650, and wireless communication by device 650. Additionally, processors 652 may include multiple instances of a processor or set of processors.

Processor 652 can communicate with a user through control interface 658 and display interface 656 coupled to a display 654. Display 654 can be, for example, a TFT (Thin-Film-Transistor Liquid Crystal Display) display or an OLED (Organic Light Emitting Diode) display, or other appropriate display technology. Display interface 656 can comprise appropriate circuitry for driving display 654 to present graphical and other information to a user (such as a UI rendering a mobile application including therapeutic content 102 of DTx 100). Control interface 658 can receive commands from a user and convert them for submission to processor 652. In addition, an external interface 662 can be provided in communication with processor 652, so as to enable near area communication of computing device 650 with other devices. External interface 662 can provide, for example, for wired communication in some implementations, or for wireless communication in other implementations, and multiple interfaces can also be used.

Memory 664 may store information within computing device 650. Memory 664 can be implemented as one or more of a computer-readable medium or media, a volatile memory unit or units, or a non-volatile memory unit or units. Expansion memory 674 can also be provided and connected to computing device 650 through expansion interface 672, which can include, for example, a SIMM (Single In Line Memory Module) card interface. Such expansion memory 674 can provide extra storage space for computing device 650, or can also store applications or other information for computing device 650. Specifically, expansion memory 674 can include instructions to carry out or supplement the processes described above, and can also include secure information. Thus, for example, expansion memory 674 can be provided as a security module for computing device 650, and can be programmed with instructions that permit secure use of computing device 650. In addition, secure applications can be provided via the SIMM cards, along with additional information, such as placing identifying information on the SIMM card in a non-hackable manner.

The memory can include, for example, flash memory and/or NVRAM memory, as discussed below. In some embodiments, a computer program product is tangibly embodied in an information carrier. The computer program product contains instructions that, when executed, perform one or more methods, such as those described above. The information carrier is a computer- or machine-readable medium, such as memory 664, expansion memory 674, or memory on processor 652 that can be received, for example, over transceiver 668 or external interface 662.

Computing device 650 can communicate wirelessly through communication interface 666, which can include digital signal processing circuitry where necessary. Communication interface 666 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 668. In addition, short-range communication can occur, such as using a Bluetooth, Wi-Fi, or other such transceiver (not shown). In addition, GPS (Global Positioning System) receiver module 670 can provide additional navigation- and location-related wireless data to computing device 650, which can be used as appropriate by applications running on computing device 650.

Computing device 650 can also communicate audibly using audio codec 660, which can receive spoken information from a user and convert it to usable digital information. Audio codec 660 can likewise generate audible sound for a user, such as through a speaker, such as in a handset of computing device 650. Such sound can include sound from voice telephone calls, can include recorded sound, such as voice messages, music files, etc. and can also include sound generated by applications operating on computing device 650.

The computing device 650 can be implemented in a number of different forms, as shown in the figure. For example, it can be implemented as a cellular telephone 680. It can also be implemented as part of a smartphone 682, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, such as magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, such as a CRT (cathode ray tube) or LCD (liquid crystal display) monitor for displaying information to the user and a keyboard and a pointing device, such as a mouse or a trackball by which the user can provide input to the computer. Other kinds of devices can be used to provide for interaction with a user as well; for example, feedback provided to the user can be any form of sensory feedback, such as visual feedback, auditory feedback, or tactile feedback; and input from the user can be received in any form, including acoustic, speech, or tactile input.

The systems and techniques described here can be implemented in a computing system that includes a back end component, such as a data server, or that includes a middleware component, such as an application server, or that includes a front end component, such as a client computer having a graphical user interface or a Web browser through which a user can interact with an implementation of the systems and techniques described here, or any combination of such back end, middleware, or front end components. The components of the system can be interconnected by any form or medium of digital data communication, such as a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

### EXAMPLE 1

### Protocol Title

A Multi-center, Randomized, Controlled Trial to Evaluate the Effectiveness of a Digital Therapeutic (CT-152) as Adjunctive Therapy in Adult Subjects Diagnosed with Major Depressive Disorder (MDD).

### CT-152

CT-152 is a digital therapeutic that delivers an interactive, software-based, cognitive, emotional, and behavioral therapy. The components of CT-152 include Emotional Faces Memory Task (EFMT), Cognitive Behavioral Therapy (CBT)-based psychotherapy lessons, and short message service (SMS) text messaging. EFMT and the psychotherapy lessons are to be completed 3 times per week over the course of 6 weeks. Text messages are sent throughout the 6-week course of treatment.

### Trial Rationale

A trial designed to test CT-152 relative to a sham in a randomized, controlled trial with an adequate sample size of subjects diagnosed with MDD who are on antidepressant therapy (ADT) monotherapy. This trial will provide data regarding the efficacy and safety of this software treatment.

### Trial Objectives and Endpoints

Primary Objective: To compare the effectiveness of CT-152 with sham, in adult subjects diagnosed with MDD who are on ADT monotherapy.

Primary efficacy endpoint: Change from baseline to Week 6 in the Montgomery-Asberg Depression Rating Scale (MADRS) total score. The durability of effect will include 3 MADRS assessments at Weeks 6, 8, and 10. In addition to demonstrating an minimal clinically important difference (MCID) group difference of 1.6 to 1.9 at Week 6 with statistical significance, durability will be demonstrated by a point estimate of the difference in change from baseline at Weeks 8 and 10 above 1.6, when comparing CT-152 and sham.

Key secondary efficacy endpoint: Change from baseline to Week 6 in Generalized Anxiety Disorder-7 (GAD-7) total score. Durability based on GAD-7 will include 3 assessments, at Weeks 6, 8, and 10, demonstrating a numerically larger improvement on point estimate of the difference in the change from baseline in GAD-7 total score at Weeks 8 and 10 in CT-152 compared to sham.

Other efficacy endpoints: Change from baseline to Weeks 2 and 4 in the MADRS total score; Change from baseline to Weeks 2 and 4 in the GAD-7 total score; MADRS response rate (≥50% reduction from baseline) at Weeks 2, 4, and 6; Change from baseline to Weeks 2, 4, and 6 in the Clinical Global Impressions-Severity (CGI-S) score; Change from baseline to Week 6 in the World Health Organization Disability Assessment Schedule (WHODAS) 2.0 total score; Change from screening to Weeks 4 and 6 in the Patient Health Questionnaire-9 (PHQ-9) total score; MADRS partial response (MADRS score reduction from baseline ≥30% and <50%) at Weeks 2, 4, and 6; MADRS response rate (full or partial, defined as ≥30% reduction in MADRS total score from baseline) at Weeks 8 and 10.

Exploratory endpoints: Remission (MADRS score ≥10 and MADRS ≥50% reduction from baseline) rates at Weeks 2, 4, and 6; Satisfaction as measured by Subject and Healthcare Professional (HCP) Satisfaction Scales; Health status as measured by the EuroQol 5-Dimension, 5-Level (EQ-5D-5L).

Safety objective: To evaluate the safety of CT-152 in adult subjects diagnosed with MDD who are on ADT monotherapy.

Safety endpoints: Frequency and severity of adverse events (AEs), serious AEs, and discontinuations from the trial due to AEs.

### Trial Design

A multi-center, randomized, controlled trial to evaluate the effectiveness of CT-152 in adult subjects diagnosed with MDD who are on ADT monotherapy for the treatment of depression. Subjects will participate in the trial for up to 13 weeks. The trial will include a screening period of up to 3 weeks, a treatment period for 6 weeks, and an extension period for 4 weeks. Eligible subjects will be randomized to 1 of 2 digital mobile applications (CT-152 or sham) on Day 1.

To mitigate subject expectation, subjects in this trial will be blinded to the efficacy hypothesis. Eligible subjects will be informed by trial site staff that a) they will participate in the trial for up to 13 weeks and will be randomized to one of two digital therapeutic treatments and b) the purpose of the trial is to compare the effectiveness of the two digital therapeutic treatments when used in addition to ADT. Both treatment arms will be presented as possibly helping to improve MDD. No references to CT-152 or sham will be made to the subject. At the conclusion of a subject's participation in the trial, and after all final visit procedures have been completed, trial site staff will inform the subject of the trial hypothesis, in other words, that one digital therapeutic was hypothesized to be more beneficial in improving depression symptoms, but there was a need for a trial to confirm. Trial site staff will be provided with debriefing guidelines to assist in this discussion with the subject.

Trial site staff will implement procedures either by telephone or by remote visit via telemedicine technology, at all visits. The screening visit may be performed in person at the discretion of the investigator. A trial site may conduct an unscheduled visit in person or remotely at any time if needed to assess a safety issue/concern.

Remote visits will be conducted using a sponsor-designated telemedicine platform with a portal accessible by the trial site staff, and subjects will be asked to download a mobile application (separate from the investigational digital mobile application) in order to provide consent to the trial and complete trial assessments, including self-administered scales.

Prior to downloading the mobile application for conducting telemedicine visits, subjects will be asked to provide consent to participate in a registry and agree to its privacy policy and terms of service required to collect subjects' information within the telemedicine platform, including identity verification. Subjects will be required to complete the identity verification process remotely before they can electronically sign the trial consent in order to comply with 21 Code of Federal Regulations (CFR) Part 11 electronic signature requirements. Details are found in the Site Operations Manual.

The screening period begins after informed consent has been obtained. Subjects who fulfill entry criteria at the screening visit will download the digital mobile application on their smartphone and receive access to an onboarding software module. A call center can assist with the downloading of the digital mobile application. During the screening period, subjects will become familiar with the software. The subject's understanding of, and interest in, the trial will be demonstrated through adequate adherence to onboarding requirements. This will be assessed by the investigator via confirmation with the subject and completion of tasks by the subject within a span of 7 consecutive days during the allotted 3-week screening window.

Following the screening visit, subjects will be considered eligible based upon the following: (1) Adherence and performance on the onboarding software module by the subject (defined as completing 3 example sessions during 7 consecutive days of the 3-week screening period and achieving a difficulty level of ≥2 in the cognitive control task by the third session). (2) Continuing to meet all inclusion and no exclusion criteria based on investigator assessment.

On Day 1, the eligible subjects will be randomized 1:1 (CT-152 or sham) across approximately 50 trial sites. The sample size at any single trial site will be capped at approximately 15% of the total subjects randomized into the trial. Randomization will be stratified by trial site.

During the treatment period (Day 1 [baseline] to Week 6) subjects will have a remote visit at Weeks 2, 4, and 6 and will be contacted by telephone by the trial site at Weeks 1, 3, and 5. Subjects will be expected to be adherent with their digital mobile application exercises during the treatment period.

After Week 6, subjects will continue participation in the trial during the extension period (Weeks 7 to 10). In the extension period, the digital mobile applications will remain installed for each group. Subjects will receive brief short message service (SMS) messages reminding subjects of the previously completed CT-152 or sham treatment courses (see the “Trial Treatment” section below for further details), and will continue their ADT. Subjects will have a remote visit at Weeks 8 and 10 and will be contacted by telephone by the trial site at Weeks 7 and 9.

The end of the trial will be Week 10.

During the treatment and extension periods, a blinded and independent expert clinical rater from a centralized vendor, who will otherwise not interact with the subject, will rate and record the Montgomery-Asberg Depression Rating Scale (MADRS) remotely by telephone while remaining blinded to treatment assignment and other clinical information. This may occur separately from the remote trial site visit but must be performed within the window described in the schedule of assessments.

The Clinical Global Impressions-Severity (CGI-S) scale will be completed by designated trial site staff at remote visits that occur during the treatment period. Other assessments to be performed during the trial include the Generalized Anxiety Disorder-7 (GAD-7), World Health Organization Disability Assessment Schedule (WHODAS) 2.0, Patient Health Questionnaire-9 (PHQ-9), Subject and Healthcare Professional (HCP) Satisfaction Scales, and the EuroQol 5-Dimension, 5-Level (EQ-5D-5L).

During the trial, the trial site staff will also administer the Columbia-Suicide Severity Rating Scale (C-SSRS), review subject adherence to the treatment sessions during the treatment period, confirm subject adherence to their current ADT, and assess adverse events (AEs) and concomitant medications.

### Trial Population

The trial will enroll male or female subjects aged 22 to 64 years old at the time of informed consent, with a current primary diagnosis of MDD based on the criteria in the Diagnostic and Statistical Manual of Mental Disorders, Fifth Edition (DSM-5), single or recurrent episode, without psychotic features and do not meet criteria for MDD with mixed features subtype, and who are on ADT monotherapy. If other allowable psychiatric diagnoses are present, they must not be considered primary (causing a higher degree of distress or impairment than MDD).

### Key Inclusion/Exclusion Criteria

In addition to the criteria mentioned under Trial Population above, key inclusion criteria are as follows:

Subjects must be in a current major depressive episode, as defined by DSM-5 criteria and confirmed by both the Mini International Neuropsychiatric Interview (M.I.N.I) and an adequate clinical psychiatric evaluation.

A score of Hamilton Rating Scale for Depression, 17-item (HAM-D17)≥18 at screening and the baseline visit (Day 1).

Subject must have a reported history for the current episode of inadequate response to their current monotherapy ADT. Treatment with the current ADT must be of adequate dose and duration, defined as at least 6 weeks at a minimum therapeutic dose (or higher) according to the Massachusetts General Hospital -Antidepressant Treatment Response Questionnaire (MGH-ATRQ) performed at screening. Inadequate response is defined as <50% reduction in depression symptom severity per the MGH-ATRQ. Additionally, the subject must be on a stable dose of their current monotherapy ADT for a minimum of 4 weeks prior to baseline (Day 1).

Subjects who are willing to maintain ADT treatment at current dose for the duration of their participation in the trial.

Subjects who are the only users of an iPhone with iPhone operating system (iOS) 13.0 or greater capabilities, or a smartphone with an Android operating system (OS) 9.0 or greater capabilities, and agree to download and use the digital mobile application as required by the protocol.

Subjects who, in the opinion of the investigator, will not require additional pharmacological intervention during the trial for the treatment of depression.

Subjects who have successfully completed the onboarding software module in the digital mobile application during the screening period.

Subjects who continue to consent to participate in the trial and are judged to understand the use of the digital mobile application at the baseline visit (Day 1).

Key exclusion criteria are as follows:

Subjects with a reported inadequate response to >1 adequate trial of ADT for the current episode. An adequate trial is defined as at least 6 weeks at a minimum therapeutic dose (or higher) according to the MGH-ATRQ. Inadequate response is defined as <50% reduction in depression symptom severity per the MGH-ATRQ.

Subjects who have been treated with psychopharmacological augmentation for depression in the past or in the current episode (such as lithium, triiodothyronine, or antipsychotics added to ADT, multiple ADTs). If, in the clinical opinion of the investigator, the subject did not receive an adequate trial of an agent used for augmentation, these subjects may be considered for inclusion following discussion and approval by the medical monitor.

Subjects who are currently receiving or have received psychotherapy within 90 days prior to screening.

Subjects who have failed to respond to an adequate course (>8 weeks) of cognitive behavioral therapy at any time in the past.

Suicidality assessment: Subjects who answer “Yes” on the C-SSRS Suicidal Ideation Item 4 (Active Suicidal Ideation with Some Intent to Act, Without Specific Plan) within the last 12 months prior to screening or at the baseline visit (Day 1), OR Subjects who answer “Yes” on the C-SSRS Suicidal Ideation Item 5 (Active Suicidal Ideation with Specific Plan and Intent) within the last 12 months prior to screening or at the baseline visit (Day 1), OR Subjects who answer “Yes” on any of the 5 C-SSRS Suicidal Behavior Items (actual attempt, interrupted attempt, aborted attempt, preparatory acts, or suicidal behavior) within the last 24 months prior to screening or at the baseline visit (Day 1), OR Subjects who, in the opinion of the investigator, present a serious risk of suicide.

Subjects who at any time in the past have been treated with electroconvulsive therapy or neuro-modulation devices (transcranial magnetic stimulation, vagus nerve stimulation, or transcranial direct current stimulation, etc.) for depression.

Subjects who at any time in the past have received ketamine, esketamine, or arketamine for treatment of depression.

Subjects who are currently using a computer, web, or smartphone software-based application or equivalent for mental health or depression. Subjects who agree to discontinue use at screening will be permitted to enter the trial.

Subjects who have a current diagnosis of substance or alcohol use disorder (excluding nicotine) per DSM-5 within 6 months prior to the screening visit.

Subjects in a current major depressive episode lasting longer than 2 years.

Subjects who are considered resistant/refractory to treatment by history and per investigator judgment.

A lifetime diagnosis of schizophrenia, schizoaffective disorder, other psychotic disorder, or Bipolar I/II disorder, or current posttraumatic stress disorder, panic disorder, or obsessive-compulsive disorder, as assessed by the M.I.N.I.

Current generalized anxiety disorder or social anxiety disorder as assessed by the M.I.N.I and considered to be primary (causing a higher degree of distress or impairment than MDD).

Subjects diagnosed with any DSM-5 personality disorder as assessed by the investigator during the psychiatric evaluation and/or from medical records.

Depression due to a general medical condition or a neurologic disorder.

History of seizure disorder other than a single childhood febrile seizure that fully resolved.

Subjects who would be likely to require prohibited concomitant therapy during the trial.

### Trial Treatment, Treatment Duration, Mode of Administration

Subjects that meet all the initial inclusion criteria and none of the exclusion criteria at screening will download and install the digital mobile application onto their own smartphone device that they will use for the trial. A dedicated call center can assist with the initial downloading of and access to the digital mobile application. The investigator will confirm the subject's understanding of, and interest in, the trial through adequate adherence to run-in onboarding requirements in the onboarding software module within a span of 7 consecutive days during the 3-week screening period (Day—21 to Day—1).

The onboarding software module will provide example cognitive control task sessions. The content of these example sessions will not include therapeutic content, so as to minimize bias once subjects are randomized to 1 of the 2 arms (CT-152 or sham).

At the baseline visit (Day 1), successful use of the onboarding software module will be confirmed. CT-152 or sham will be activated within the digital mobile application during the baseline visit with an access code.

CT-152 delivers an interactive, software-based intervention featuring cognitive-emotional training, psychotherapy lessons, psychotherapy messages, and engagement messages. Each treatment session will consist of an Emotional Faces Memory Task (EFMT) exercise and a psychotherapy lesson. Sham will serve as a control.

Sham will provide a cognitive training exercise designed to retain user interest while minimizing any therapeutic effect. Each treatment session will consist of a Shapes Memory Task (SMT) exercise. It will present users with an analogous structure, matched for time and attention to the cognitive-emotional training exercise found in CT-152. In order to retain the intended placebo nature of the sham, it will not include EFMT or psychotherapy content.

Subjects will participate in the trial for up to 13 weeks. This includes a screening period of up to 3 weeks; due to the onboarding adherence requirement, a minimum of 7 consecutive days will be required for screening. Extensions to the screening period, if requested by the investigator, may be granted after discussion and approval by the medical monitor.

The intervention will begin the same day as the baseline visit, once the baseline visit has been completed. The subjects will progress through a treatment schedule of 18 sessions (approximately 30-45 minutes) at a rate of 3 sessions per week over the 6-week treatment period (Day 1 [baseline] to Week 6).

After Week 6, subjects will continue participation in the trial during the extension period (Weeks 7 to 10). In the extension period, the digital mobile applications will remain installed for each group with EFMT and SMT no longer available. Psychotherapy content provided previously will remain available for optional reference in the CT-152 group but no new therapeutic content will be introduced and no required treatment schedule is in place. The 2 groups will each receive brief SMS messages in the extension period reminding subjects of the previously completed CT-152 and sham treatment courses.

A dedicated call center is available to support the subject and the trial site on the initial downloading of and access to the digital mobile application, as well as any technical issues with the digital mobile application throughout the trial.

Subjects must be instructed by the investigator to contact the call center with any technical questions about the digital mobile application. All calls to the call center will be documented and processed. Basic user technical issues will be resolved by the call center.

If a subject contacts the call center with an AE, the call center will log the call and will immediately provide this information to the trial site and sponsor or sponsor's designee for follow-up.

If a subject contacts the call center with a possible or suspected product quality complaint (PQC), the call center will log the call. All call records (tickets) captured by the call center will be provided to the Click Therapeutics Quality Team for PQC analysis, tracking, and resolution.

If a subject reports a possible or suspected PQC to the investigator or designee during a remote visit or telephone contact, the investigator or designee is to immediately contact the call center, which will log the call and immediately provide the information to the Click Therapeutics Quality Team.

In addition to call tracking, calls with the call center may be recorded for quality purposes. Call center contact information and processes are detailed in the Site Operations Manual.

### Trial Assessments

Assessments for Efficacy: MADRS, GAD-7, CGI-S, WHODAS 2.0, and PHQ-9.

Assessments for Safety: AEs (including AEs related to the worsening of depressive symptoms) and C-SSRS.

Screening/Other: Subject and HCP Satisfaction Scales, EQ-5D-5L, M.I.N.I, HAM-D17, Antidepressant Treatment Response Questionnaire, DSM-5 diagnosis of MDD, urine drugs-of-abuse screen, pregnancy test, and adherence check.

### Statistical Methods

The initial sample size is calculated to detect a 3-unit difference between CT-152+ADT and sham+ADT in the change from baseline in MADRS total score with 85% power at a 2-sided α=0.05 level, assuming a common standard deviation of 9. The resulting sample size is 324 evaluable subjects in total (162 subjects in each arm). To compensate for subjects that fail to have evaluable assessments of MADRS total score in the full analysis set (FAS) sample (estimated at up to 10% of all subjects), a total of 360 subjects (180 subjects in each arm) will be randomized in this trial.

Due to the limitations of applying assumptions on the treatment effect size, and in order to ensure adequate power of the trial, an unblinded interim analysis will be conducted by a DMC. The final sample size could be increased to 540 subjects (270 subjects in each arm) as per recommendation of the DMC. Using the O'Brien-Fleming spending function, a significance level of 0.003 (2-sided) is allocated to this interim analysis. The corresponding final significance level is 0.049 (2-sided).

The null hypothesis of the statistical test comparing CT-152+ADT and sham+ADT, based on the primary efficacy endpoint, is that the change in MADRS using CT-152+ADT is equal to the change in MADRS using the sham+ADT.

The primary analysis will be conducted on the change from baseline in MADRS total score to the final on-therapy evaluation (Week 6) based on the FAS sample adjusted for the baseline MADRS total score.

A minimal clinically important difference (MCID) range for the MADRS is considered to be between 1.6 and 1.9. In this trial, we plan to detect a 3-point difference on the primary efficacy endpoint between the treatment groups. The 3-point treatment difference is above the MCID range. This trial is considered positive if the trial will be stopped at the interim analysis for efficacy, or if the p-value of the statistical comparison based on the primary efficacy endpoint at final is <0.049.

The primary analysis will utilize mixed model repeated measurements (MMRM) with treatment, visit, treatment by visit interaction, and site as fixed effects to assess heterogeneity of treatment effects. The key secondary efficacy endpoint and other efficacy endpoints will be analyzed based on the FAS as described for the primary analysis.

The null hypothesis of the statistical test comparing CT-152+ADT and sham+ADT, based on the key secondary efficacy endpoint, is that the change from baseline to Week 6 in the GAD-7 total score using CT-152+ADT is equal to the change from baseline to Week 6 in the GAD-7 total score using sham+ADT.

The key secondary efficacy endpoint will be analyzed using the same method (MMRM) as in the primary analysis with a replacement of the interaction term of visit by baseline GAD-7 total score as a covariate.

The durability of effect of CT-152 will be assessed based on MADRS total score and GAD-7 total score at Weeks 6, 8 and 10. Change from baseline to Week 8 and 10 in the above assessments will be analyzed using MMRM as described for the primary analysis.

An unblinded interim analysis of efficacy data will be conducted on approximately the first 180 randomized subjects. The unblinded interim analysis will be carried out when these subjects have either completed the Week 6 visit or discontinued prior to Week 6.

The difference between CT-152 and sham based on the primary efficacy endpoint will be tested at the unblinded interim analysis. The sample size will be re-estimated only based on the conditional power determined at the interim analysis. The adaptive designs methodology published by Chen, DeMets, and Lan will be used to increase the sample size based on an interim estimate of the treatment effect size, possibly combined with other external information, without inflating the type I error.

Examples of various enumerated embodiments include: 1. A method for treating depression, the method comprising: providing memory task exercises according to a first schedule, wherein providing the memory task exercises comprises: sequentially displaying a first plurality of expression images to a patient receiving treatment for depression, wherein each of the first plurality of expression images is configured to convey a respective emotion; prompting the patient to provide an input indicating whether the respective emotion of a first expression image of the first plurality of expression images matches the respective emotion of a second expression image of the first plurality of expression images; and receiving a patient response indicating whether the respective emotions of the first and second expression images of the first plurality of expression images match one another; providing psychotherapy lessons according to a second schedule, wherein providing the psychotherapy lessons includes displaying an animated video to the patient that is configured to provide therapeutic intervention through at least one of emotion regulation, behavioral activation and cognitive restructuring, wherein the first and second schedules define a six-week treatment period.
2. The method of embodiment 1, wherein: the first and second schedules include providing the memory task exercises and the psychotherapy lessons at least three days per week, respectively, and the memory task exercises and the psychotherapy lessons are provided on alternating days of the week.
3. The method of any one of embodiments 1-2, wherein the psychotherapy lessons comprise cognitive behavioral therapy lessons, each having a duration of approximately 3-5 minutes.
4. The method of any one of embodiments 1-3, wherein one or more of the psychotherapy lessons include an activity or task to be completed by the patient. The method of any one of embodiments 1-4, wherein providing the psychotherapy lessons according to the second schedule comprises: rendering a first plurality of animated videos configured to provide therapeutic intervention through emotion regulation during a first week of the six-week treatment period; rendering a second plurality of animated videos configured to provide therapeutic intervention through behavioral activation during a second week of the six-week treatment period; rendering a third plurality of animated videos configured to provide therapeutic intervention through cognitive restructuring during a third week of the six-week treatment period; rendering a fourth plurality of animated videos configured to provide therapeutic intervention through emotion regulation during a fourth week of the six-week treatment period; rendering a fifth plurality of animated videos configured to provide therapeutic intervention through behavioral activation during a fifth week of the six-week treatment period; and rendering a sixth plurality of animated videos configured to provide therapeutic intervention through cognitive restructuring during a sixth week of the six-week treatment period.
6. The method of any one of embodiments 1-5, wherein at least one of the psychotherapy lessons is configured to reduce a severity of depression as measured by the Montgomery-Asberg Depression Rating Scale or the Hamilton Depression Rating Scale.
7. The method of any one of embodiments 1-6, wherein: the first plurality of expression images comprise facial expressions, and each of the respective emotions of the first plurality of expression images are configured to represent at least one of: happiness, worry, anger, sadness, surprise, or disgust.
8. The method of any one of embodiments 1-7, wherein providing the memory task exercises according to the first schedule further comprises: determining a score based at least in part on whether the patient response is correct.
9. The method of embodiment 8, wherein providing the memory task exercises according to the first schedule further comprises: sequentially displaying a second plurality of expression images to the patient, wherein each of the second plurality of expression images is configured to convey a respective emotion; and prompting the patient to provide an input indicating whether the respective emotion of a first expression image of the second plurality of expression images matches the respective emotion of a second expression image of the second plurality of expression images, wherein the second expression image of the second plurality of expression images is N-images back from the first expression image of the second plurality of expression images.
10. The method of embodiment 9, wherein a value of N is an integer configured to be adjusted based at least in part on whether the patient response with respect to the first plurality of expression images was correct.
11. The method of any one of embodiments 9-10, wherein the first and second expression images of the second plurality of expression images each include a respective emotional intensity configured to be adjusted based at least in part on whether the patient response with respect to the first plurality of expression images was correct.
12. A non-transitory computer-readable medium storing computer program instructions that, when executed by one or more processors of a computing device, effectuate operations comprising any one of embodiments 1-11.
13. A medical treatment device comprising: memory storing computer program instructions; and one or more processors configured to execute the computer program instructions to effectuate operations comprising any one of embodiments 1-11.
14. A medical treatment system comprising: memory storing computer program instructions; and one or more processors configured to execute the computer program instructions to effectuate operations comprising any one of embodiments 1-11.
15. The medical treatment system of embodiment 14, further comprising: a plurality of user devices, wherein the user device is one of the plurality of user devices.
16. A treatment device to treat a mood disorder via a digital therapeutic comprising a plurality of memory task exercises and a plurality of psychotherapy lessons, the treatment device comprising: a processor programmed to access a treatment schedule that defines a duration and timing of administration of the digital therapeutic, identify, on a first day of the treatment schedule, a first memory task exercise, from among the plurality of memory task exercises, to be administered on the first day, access a first plurality of expression images associated with the first memory task exercise, wherein each of the first plurality of expression images is configured to convey a respective emotion, sequentially display, via the display device, the first plurality of expression images to a patient receiving treatment for the mood disorder, display a prompt to the patient to provide an input on whether the respective emotion of a first expression image of the first plurality of expression images matches the respective emotion of a second expression image of the first plurality of expression images, receive a patient response indicating whether the respective emotions of the first and second expression images of the first plurality of expression images match one another, identify, on a second day of the treatment schedule, a psychotherapy lesson from among the plurality of psychotherapy lessons, to be administered on the second day, and display the psychotherapy lesson with an option to repeat one or more prior psychotherapy lessons. The treatment schedule may be accessed from local memory or may be transmitted from an application server.
17. The treatment device of embodiment 16, wherein the treatment schedule comprises a first treatment schedule for the plurality of memory task exercises and a second treatment schedule for the psychotherapy lessons.
18. The treatment device of embodiment 16, wherein the second day is immediately after the first day.
19. The treatment device of embodiment 16, wherein the treatment schedule defines a treatment period of at least six weeks.
20. The treatment device of embodiment 16, wherein the treatment schedule defines alternating days of a memory task exercise and a psychotherapy lessons throughout a duration of a treatment period.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (for example, within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (such as content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (in other words, meaning having the potential to), rather than the mandatory sense (in other words, meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, in other words, encompassing both “and” and “or.” Terms describing conditional relationships, such as “in response to X, Y,” “upon X, Y,” “if X, Y,” “when X, Y,” and the like, encompass causal relationships in which the antecedent is a necessary causal condition, the antecedent is a sufficient causal condition, or the antecedent is a contributory causal condition of the consequent, such as “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, such as the antecedent is relevant to the likelihood of the consequent occurring. Statements in which a plurality of attributes or functions are mapped to a plurality of objects (such as one or more processors performing steps A, B, C, and D) encompasses both all such attributes or functions being mapped to all such objects and subsets of the attributes or functions being mapped to subsets of the attributes or functions (such as both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, in other words, each does not necessarily mean each and every. Limitations as to sequence of recited steps should not be read into the claims unless explicitly specified, such as with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (such as “at least Z of A, B, or C), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, such as reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, such as text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively.

Those skilled in the art will recognize that the present teachings are amenable to a variety of modifications and/or enhancements. For example, although the implementation of various components described above may be embodied in a hardware device, it may also be implemented as a software only solution—such as an installation on an existing server. In addition, the conversation management techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

What is claimed is:

1. A treatment device to treat a mood disorder via a digital therapeutic comprising a plurality of memory task exercises and a plurality of psychotherapy lessons, the treatment device comprising:
a processor programmed to:
access a treatment schedule that defines a duration and timing of administration of the digital therapeutic;
identify, on a first day of the treatment schedule, a first memory task exercise, from among the plurality of memory task exercises, to be administered on the first day;
access a first plurality of expression images associated with the first memory task exercise, wherein each of the first plurality of expression images is configured to convey a respective emotion;
sequentially display, via the display device, the first plurality of expression images to a patient receiving treatment for the mood disorder;
display a prompt to the patient to provide an input on whether the respective emotion of a first expression image of the first plurality of expression images matches the respective emotion of a second expression image of the first plurality of expression images;
receive a patient response indicating whether the respective emotions of the first and second expression images of the first plurality of expression images match one another;
identify, on a second day of the treatment schedule, a psychotherapy lesson from among the plurality of psychotherapy lessons, to be administered on the second day; and
display the psychotherapy lesson with an option to repeat one or more prior psychotherapy lessons.

2. The treatment device of claim 1, wherein the treatment schedule comprises a first treatment schedule for the plurality of memory task exercises and a second treatment schedule for the psychotherapy lessons.

3. The treatment device of claim 1, wherein the second day is immediately after the first day.

4. The treatment device of claim 1, wherein the second day is immediately before the first day.

5. The treatment device of claim 1, wherein the treatment schedule defines a treatment period of at least six weeks.

6. The treatment device of claim 1, wherein the treatment schedule defines alternating days of a memory task exercise and a psychotherapy lessons throughout a duration of a treatment period.

7. A method for treating depression, the method comprising:
providing memory task exercises according to a first schedule, wherein providing the memory task exercises comprises:
sequentially displaying a first plurality of expression images to a patient receiving treatment for depression, wherein each of the first plurality of expression images is configured to convey a respective emotion;
prompting the patient to provide an input indicating whether the respective emotion of a first expression image of the first plurality of expression images matches the respective emotion of a second expression image of the first plurality of expression images; and
receiving a patient response indicating whether the respective emotions of the first and second expression images of the first plurality of expression images match one another; and
providing psychotherapy lessons according to a second schedule, wherein providing the psychotherapy lessons includes displaying an animated video to the patient that is configured to provide therapeutic intervention through at least one of emotion regulation, behavioral activation and cognitive restructuring.

8. The method of claim 7, wherein:
the first and second schedules include providing the memory task exercises and the psychotherapy lessons at least three days per week, respectively, and
the memory task exercises and the psychotherapy lessons are provided on alternating days of the week.

9. The method of claim 7, wherein the psychotherapy lessons comprise cognitive behavioral therapy lessons, each having a duration of approximately 3-5 minutes.

10. The method of claim 7, wherein one or more of the psychotherapy lessons include an activity or task to be completed by the patient.

11. The method of claim 7, wherein providing the psychotherapy lessons according to the second schedule comprises:
rendering a first plurality of animated videos configured to provide therapeutic intervention through emotion regulation during a first week of the six-week treatment period;
rendering a second plurality of animated videos configured to provide therapeutic intervention through behavioral activation during a second week of the six-week treatment period;
rendering a third plurality of animated videos configured to provide therapeutic intervention through cognitive restructuring during a third week of the six-week treatment period;
rendering a fourth plurality of animated videos configured to provide therapeutic intervention through emotion regulation during a fourth week of the six-week treatment period;
rendering a fifth plurality of animated videos configured to provide therapeutic intervention through behavioral activation during a fifth week of the six-week treatment period; and
rendering a sixth plurality of animated videos configured to provide therapeutic intervention through cognitive restructuring during a sixth week of the six-week treatment period.

12. The method of claim 7, wherein at least one of the psychotherapy lessons is configured to reduce a severity of depression as measured by the Montgomery-Asberg Depression Rating Scale or the Hamilton Depression Rating Scale.

13. The method of claim 7, wherein:
the first plurality of expression images comprise facial expressions, and
each of the respective emotions of the first plurality of expression images are configured to represent at least one of: happiness, worry, anger, sadness, surprise, or disgust.

14. The method of claim 7, wherein providing the memory task exercises according to the first schedule further comprises:
determining a score based at least in part on whether the patient response is correct.

15. The method of claim 14, wherein providing the memory task exercises according to the first schedule further comprises:
sequentially displaying a second plurality of expression images to the patient, wherein each of the second plurality of expression images is configured to convey a respective emotion; and
prompting the patient to provide an input indicating whether the respective emotion of a first expression image of the second plurality of expression images matches the respective emotion of a second expression image of the second plurality of expression images,
wherein the second expression image of the second plurality of expression images is N-images back from the first expression image of the second plurality of expression images.

16. The method of claim 15, wherein a value of N is an integer configured to be adjusted based at least in part on whether the patient response with respect to the first plurality of expression images was correct.

17. The method of claim 15, wherein the first and second expression images of the second plurality of expression images each include a respective emotional intensity configured to be adjusted based at least in part on whether the patient response with respect to the first plurality of expression images was correct.

18. The method of claim 7, wherein the first and second schedules define a six-week treatment period.

19. A computer-readable medium for storing instructions that render therapeutic content for treating depression, the instructions being executable by one or more processing devices to perform operations comprising:
providing memory task exercises according to a first schedule, wherein providing the memory task exercises includes:
sequentially displaying a first plurality of expression images to a patient receiving treatment for depression, wherein each of the first plurality of expression images is configured to convey a respective emotion;
prompting the patient to provide an input indicating whether the respective emotion of a first expression image of the first plurality of expression images matches the respective emotion of a second expression image of the first plurality of expression images; and
receiving a patient response indicating whether the respective emotions of the first and second expression images of the first plurality of expression images match one another; and
providing psychotherapy lessons according to a second schedule, wherein providing the psychotherapy lessons includes displaying an animated video to the patient that is configured to provide therapeutic intervention through at least one of emotion regulation, behavioral activation and cognitive restructuring.

20. The computer-readable medium of claim 19, wherein:
the first and second schedules each include providing the memory task exercises and the psychotherapy lessons three days per week, respectively, and
the memory task exercises and the psychotherapy lessons are provided on alternating days of the week.

21. The computer-readable medium of claim 19, wherein the psychotherapy lessons comprise cognitive behavioral therapy, each having a duration of approximately 3-5 minutes.

22. The computer-readable medium of claim 19, wherein one or more of the psychotherapy lessons include an activity or task to be completed by the patient.

23. The computer-readable medium of claim 19, wherein providing the psychotherapy lessons according to the second schedule comprises:
rendering a first plurality of animated videos configured to provide therapeutic intervention through emotion regulation during a first week of the six-week treatment period;
rendering a second plurality of animated videos configured to provide therapeutic intervention through behavioral activation during a second week of the six-week treatment period;
rendering a third plurality of animated videos configured to provide therapeutic intervention through cognitive restructuring during a third week of the six-week treatment period;
rendering a fourth plurality of animated videos configured to provide therapeutic intervention through emotion regulation during a fourth week of the six-week treatment period;
rendering a fifth plurality of animated videos configured to provide therapeutic intervention through behavioral activation during a fifth week of the six-week treatment period; and
rendering a sixth plurality of animated videos configured to provide therapeutic intervention through cognitive restructuring during a sixth week of the six-week treatment period.

24. The computer-readable medium of claim 19, wherein at least one of the psychotherapy lessons is configured to reduce a severity of depression as measured by the Montgomery-Asberg Depression Rating Scale or the Hamilton Depression Rating Scale.

25. The computer-readable medium of claim 19, wherein:
the first plurality of expression images comprise facial expressions, and
each of the respective emotions of the first plurality of expression images are configured to represent one of happy, worried, angry, sad, surprised and disgusted.

26. The computer-readable medium of claim 19, wherein providing the memory task exercises according to the first schedule further comprises determining a score based at least in part on whether the patient response is correct.

27. The computer-readable medium of claim 19, wherein providing the memory task exercises according to the first schedule further comprises:
sequentially displaying a second plurality of expression images to the patient, wherein each of the second plurality of expression images is configured to convey a respective emotion; and
prompting the patient to provide an input indicating whether the respective emotion of a first expression image of the second plurality of expression images matches the respective emotion of a second expression image of the second plurality of expression images,
wherein the second expression image of the second plurality of expression images is N-images back from the first expression image of the second plurality of expression images.

28. The computer-readable medium of claim 27, wherein a value of N is an integer configured to be adjusted based at least in part on whether the patient response with respect to the first plurality of expression images was correct.

29. The computer-readable medium of claim 27, wherein the first and second expression images of the second plurality of expression images each include a respective emotional intensity configured to be adjusted based at least in part on whether the patient response with respect to the first plurality of expression images was correct.

30. The computer-readable medium of claim 19, wherein the first and second schedules define a six-week treatment period.
